(19) United States
(12) Patent Application Publication
(10) Pub. No.: US 2019/0290597 A1
(43) Pub. Date: Sep. 26, 2019

(54) DERMAL AND TRANSDERMAL ADMINISTRATION OF TREPROSTINIL AND SALTS THEREOF

(71) Applicant: CORSAIR PHARMA, INC., SOUTH SAN FRANCISCO, CA (US)

(72) Inventors: Xiaoming ZHANG, Sunnyvale, CA (US); Cyrus K. BECKER, Pleasanton, CA (US); Meenakshi S. VENKATRAMAN, Fremont, CA (US)

(21) Appl. No.: 16/465,683

(22) PCT Filed: Dec. 5, 2017
(86) PCT No.: PCT/US2017/064612

§ 371 Date: May 31, 2019

Related U.S. Application Data
(60) Provisional application No. 62/430,053, filed on Dec. 5, 2016.

(51) Int. Cl. A61K 9/70; A61K 31/5575; A61K 9/00; A61K 47/10; A61K 47/14; A61K 47/20; A61K 47/22

(52) U.S. Cl. CPC A61K 9/7084; A61K 31/5575; A61K 9/7092; A61K 47/22; A61K 47/10; A61K 47/14; A61K 47/20; A61K 9/0014

(57) ABSTRACT

The present disclosure provides methods, compositions, devices and systems for dermal and transdermal administration of treprostinil or salts thereof, and optionally an additional therapeutic agent. Treprostinil and salts thereof can be dermally or transdermally administered to treat any medical conditions responsive to treatment with treprostinil, including pulmonary hypertension, such as pulmonary arterial hypertension.

### CROSS-REFERENCE TO RELATED APPLICATIONS

This application claims priority to and the benefit of U.S. Provisional Application No. 62/430,053, which was filed on Dec. 5, 2016 and whose entire disclosure is incorporated herein by reference for all purposes.

### BACKGROUND OF THE DISCLOSURE

Pulmonary hypertension (PH), which includes pulmonary arterial hypertension (PAH), is a disease that can result in death and is characterized by increased pulmonary artery pressure and pulmonary vascular resistance. Some drugs that can be used to treat PH or PAH cannot be effectively administered orally for various reasons and are generally administered via subcutaneous, intravenous or intramuscular routes. These routes of administration generally require intervention by a healthcare professional, and can entail considerable discomfort as well as potential local trauma to the patient.

One example of such a drug is treprostinil. Treprostinil as the free acid has an absolute oral bioavailability of less than 10% and a very short systemic half-life due to significant metabolism. Treprostinil can presently be administered by intravenous or subcutaneous injection, which has drawbacks such as pain at the site of injection and risk of infection. Treprostinil can also presently be administered by inhalation, but about 50% of PAH patients cannot take inhaled treprostinil due to irritation to the lungs, and treprostinil administered by inhalation exhibits a large peak-to-trough ratio of plasma concentrations. Treprostinil has the structure shown below. Treprostinil can exist as a free carboxylic acid or as a salt, such as a sodium or diethanolamine salt.

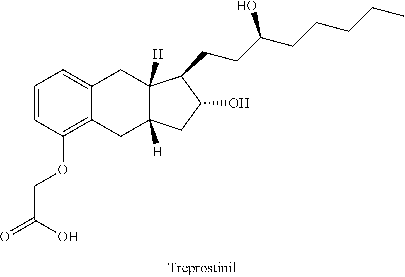 [SMILES: CCCCC[C@H](O)CC[C@@H]1[C@H]2Cc3cccc(OCC(=O)O)c3C[C@H]2C[C@H]1O]

### SUMMARY OF THE DISCLOSURE

The present disclosure describes methods, compositions, devices and systems for dermal and transdermal administration of treprostinil and salts thereof, which can provide increased local or systemic availability of treprostinil and salts thereof. Without limiting the foregoing, in some embodiments, the methods, compositions, devices and systems do not cause significant or excessive skin irritation. A treprostinil-containing composition, device or system can optionally include an additional therapeutic agent. In some embodiments, a treprostinil-containing composition, device or system is applied to the surface of the skin. In certain embodiments, treprostinil or a salt thereof (e.g., treprostinil sodium or treprostinil diethanolamine) is administered via a transdermal patch. A transdermal patch can be designed to deliver treprostinil or a salt thereof passively (passive transport of treprostinil as the carboxylic acid in some embodiments), optionally with the use of one or more chemical permeation enhancers, or actively with the assistance of a physical enhancement technique (e.g., an iontophoretic patch, a sonophoretic patch, a microneedles patch or a patch to which high pressure is applied).

The dermally or transdermally administered treprostinil or a salt thereof can be used to treat any medical conditions responsive to treatment with treprostinil. An additional therapeutic agent can optionally be used in combination with treprostinil or a salt thereof to treat a medical condition. In certain embodiments, treprostinil or a salt thereof is dermally or transdermally administered to treat pulmonary hypertension, such as PAH.

### BRIEF DESCRIPTION OF THE DRAWINGS

A better understanding of features and advantages of the present disclosure will be obtained by reference to the following detailed description, which sets forth illustrative embodiments of the disclosure, and the accompanying drawings.

FIG. 1 illustrates a cross-section of an embodiment of a reservoir-type transdermal patch.

FIG. 2 depicts a cross-section of an embodiment of a matrix-type transdermal patch.

### DETAILED DESCRIPTION OF THE DISCLOSURE

While various embodiments of the present disclosure are described herein, it will be obvious to those skilled in the art that such embodiments are provided by way of example only. Numerous modifications and changes to, and variations and substitutions of, the embodiments described herein will be apparent to those skilled in the art without departing from the disclosure. It is understood that various alternatives to the embodiments described herein can be employed in practicing the disclosure. It is also understood that every embodiment of the disclosure can optionally be combined with any one or more of the other embodiments described herein which are consistent with that embodiment.

Where elements are presented in list format (e.g., in a Markush group), it is understood that each possible subgroup of the elements is also disclosed, and any one or more elements can be removed from the list or group.

It is also understood that, unless clearly indicated to the contrary, in any method described or claimed herein that includes more than one act or step, the order of the acts or steps of the method is not necessarily limited to the order in which the acts or steps of the method are recited, but the disclosure encompasses embodiments in which the order is so limited.

It is further understood that, in general, where an embodiment in the description or the claims is referred to as comprising one or more features, the disclosure also encompasses embodiments that consist of, or consist essentially of, such feature(s).

It is also understood that any embodiment of the disclosure, e.g., any embodiment found within the prior art, can be explicitly excluded from the claims, regardless of whether or not the specific exclusion is recited in the specification.

Headings are included herein for reference and to aid in locating certain sections. Headings are not intended to limit the scope of the embodiments and concepts described in the sections under those headings, and those embodiments and concepts may have applicability in other sections throughout the entire disclosure.

All patent literature and all non-patent literature cited herein are incorporated herein by reference in their entirety to the same extent as if each patent literature or non-patent literature were specifically and individually indicated to be incorporated herein by reference in its entirety.

### I. DEFINITIONS

As used in the specification and the appended claims, the indefinite articles “a” and “an” and the definite article “the” can include plural referents as well as singular referents unless specifically stated otherwise or the context clearly indicates otherwise.

The term “about” or “approximately” means an acceptable error for a particular value as determined by one of ordinary skill in the art, which depends in part on how the value is measured or determined. In certain embodiments, the term “about” or “approximately” means within one standard deviation. In some embodiments, when no particular margin of error (e.g., a standard deviation to a mean value given in a chart or table of data) is recited, the term “about” or “approximately” means that range which would encompass the recited value and the range which would be included by rounding up or down to the recited value as well, taking into account significant figures. In certain embodiments, the term “about” or “approximately” means within ±20%, 15%, 10% or 5% of the specified value. Whenever the term “about” or “approximately” precedes the first numerical value in a series of two or more numerical values or in a series of two or more ranges of numerical values, the term “about” or “approximately” applies to each one of the numerical values in that series of numerical values or in that series of ranges of numerical values.

Whenever the term “at least” or “greater than” precedes the first numerical value in a series of two or more numerical values, the term “at least” or “greater than” applies to each one of the numerical values in that series of numerical values.

Whenever the term “no more than” or “less than” precedes the first numerical value in a series of two or more numerical values, the term “no more than” or “less than” applies to each one of the numerical values in that series of numerical values.

The term “pharmaceutically acceptable” refers to a substance (e.g., an active ingredient or an excipient) that is suitable for use in contact with the tissues and organs of a subject without excessive irritation, allergic response, immunogenicity and toxicity, is commensurate with a reasonable benefit/risk ratio, and is effective for its intended use. A “pharmaceutically acceptable” excipient or carrier of a pharmaceutical composition is also compatible with the other ingredients of the composition.

The term “therapeutically effective amount” refers to an amount of a compound that, when administered to a subject, is sufficient to prevent development of, or to alleviate to some extent, the medical condition being treated or one or more symptoms associated with the condition. The term “therapeutically effective amount” also refers to an amount of a compound that is sufficient to elicit the biological or medical response of a cell, tissue, organ, system, animal or human which is sought by a researcher, veterinarian, medical doctor or clinician.

The terms “treat”, “treating”, and “treatment” include alleviating or abrogating a medical condition or one or more symptoms associated with the condition, and alleviating or eradicating one or more causes of the condition. Reference to “treatment” of a condition is intended to include prevention of the condition. The terms “prevent”, “preventing”, and “prevention” include precluding or delaying the onset of a medical condition or one or more symptoms associated with the condition, precluding a subject from acquiring a condition, and reducing a subject's risk of acquiring a condition. The term “medical conditions” includes diseases and disorders.

The term “subject” refers to an animal, including but not limited to a mammal, such as a primate (e.g., a human, a chimpanzee or a monkey), a rodent (e.g., a rat, a mouse, a guinea pig, a gerbil or a hamster), a lagomorph (e.g., a rabbit), a swine (e.g., a pig), an equine (e.g., a horse), a canine (e.g., a dog) or a feline (e.g., a cat). The terms “subject” and “patient” are used interchangeably herein in reference to, e.g., a mammalian subject, such as a human subject.

The term “compound” (including “treprostinil”) encompasses salts, solvates, hydrates, clathrates and polymorphs of that compound. A “solvate” of a compound includes a stoichiometric or non-stoichiometric amount of a solvent (e.g., water, acetone or an alcohol [e.g., ethanol]) bound non-covalently to the compound. A “hydrate” of a compound includes a stoichiometric or non-stoichiometric amount of water bound non-covalently to the compound. A “clathrate” of a compound contains molecules of a substance (e.g., a solvent) enclosed in the crystal structure of the compound. A “polymorph” of a compound is a crystalline form of the compound. The specific recitation of “salt”, “solvate”, “hydrate”, “clathrate” or “polymorph” with respect to a compound (e.g., treprostinil) in certain instances of the disclosure shall not be interpreted as an intended omission of any of these forms in other instances of the disclosure where the term “compound” (including “treprostinil”) is used without recitation of any of these forms, unless the context clearly indicates otherwise.

### II. STEREOISOMERS

It is understood that the present disclosure encompasses all possible stereoisomers, including all possible diastereomers and both enantiomers in substantially pure form and mixtures of two or more diastereomers in any ratio and mixtures of both enantiomers in any ratio (including a racemic mixture of enantiomers), of the compounds described herein (including treprostinil), and not only the specific stereoisomers as indicated by drawn structure or nomenclature. Some embodiments of the disclosure relate to the specific stereoisomers indicated by drawn structure or nomenclature. If the phrase “or stereoisomers thereof” or the like with respect to a compound (e.g., treprostinil) is specifically recited in certain instances of the disclosure, that shall not be interpreted as an intended omission of any of the other possible stereoisomers of the compound in other instances of the disclosure where the term “compound” (including “treprostinil”) is used without recitation of the phrase “or stereoisomers thereof” or the like, unless the context clearly indicates otherwise.

### III. SALT FORMS OF COMPOUNDS

The compounds described herein (including treprostinil) may exist or be used in the form of a pharmaceutically acceptable salt. For example, treprostinil has a carboxyl group, and thus can form an addition salt with a base. Pharmaceutically acceptable base addition salts can be formed with, e.g., metals (e.g., alkali metals or alkaline earth metals) or amines (e.g., organic amines). Examples of metals useful as cations include without limitation alkali metals (e.g., lithium, sodium, potassium and cesium), alkaline earth metals (e.g., magnesium, calcium and barium), aluminum and zinc. Metal cations can be provided by way of, e.g., inorganic bases, such as hydroxides, carbonates and hydrogen carbonates. Non-limiting examples of organic amines useful for forming base addition salts include 2-amino-2-methyl-1,3-propanediol, chloroprocaine, choline, cyclohexylamine, dibenzylamine, N,N′-dibenzylethylenediamine, dicyclohexylamine, diethanolamine, ethylenediamine, N-ethylpiperidine, histidine, isopropylamine, N-methylglucamine, procaine, pyrazine, triethanolamine, triethylamine, trimethylamine, and tris(hydroxymethyl)aminomethane (trometamol or tromethamine).

In some embodiments, treprostinil is used or administered in the form of a salt (e.g., when iontophoresis is employed). In certain embodiments, treprostinil is used or administered as an alkali metal salt, such as treprostinil sodium. In other embodiments, treprostinil is used or administered as an amine salt, such as treprostinil diethanolamine, treprostinil triethanolamine, treprostinil 2-amino-2-methyl-1,3-propanediol or treprostinil tris(hydroxymethyl)aminomethane.

If a compound has a basic atom or functional group (e.g., a basic nitrogen atom), the compound can form an addition salt with an acid. Non-limiting examples of acids useful for forming acid addition salts include mineral acids (e.g., HCl, HBr, HI, nitric acid, phosphoric acid and sulfuric acid) and organic acids, such as carboxylic acids (e.g., acetic acid) and sulfonic acids (e.g., ethanesulfonic acid). Pharmaceutically acceptable salts are discussed in detail in Handbook of Pharmaceutical Salts, Properties, Selection and Use, P. Stahl and C. Wermuth, Eds., Wiley-VCH (2011).

### IV. POLYMORPHS OF TREPROSTINIL

Polymorphs of treprostinil and salts thereof are known in the art. For example, crystalline Form A of treprostinil as described in US 2015/0005384 has characteristic peaks at the following 20 angles in the X-ray powder diffraction (XRPD) pattern: 3.0±0.2°, 13.5±0.2°, 17.3±0.2°, 18.6±0.2°, and 21.7±0.2°. As another example, crystalline Form B of treprostinil as described in US 2015/0011637 has characteristic peaks at the following 20 angles in the XRPD pattern: 2.9±0.2°, 6.6±0.2°, 12.6±0.2°, 13.2±0.2°, 18.0±0.2°, 18.8±0.2°, 20.7±0.2°, 21.4±0.2°, 22.2±0.2°, 23.1±0.2°, and 25.3±0.2°.

In addition, US 2014/0275262 describes crystalline treprostinil monohydrate Forms A and B and crystalline anhydrous treprostinil Form C. Crystalline treprostinil monohydrate Form A is characterized by an XRPD pattern comprising peaks at 5.2, 10.4, 11.6, 12.6, 16.2, 20.0, 21.7 and 22.7°2θ±0.2°2θ. Crystalline treprostinil monohydrate Form B is characterized by an XRPD pattern comprising peaks at 5.3, 10.7, 12.1, 19.4, 20.6, 21.6, 22.3 and 24.4°2θ±0.2°2θ. Crystalline anhydrous treprostinil Form C is characterized by an XRPD pattern comprising peaks at 6.55 and 20.7°2θ±0.2°2θ, and by a differential scanning calorimetry curve having a minor endotherm at about 78.3° C. and a major endotherm at about 126.3° C.

U.S. Pat. No. 8,350,079 also discloses a crystalline treprostinil monohydrate form. Furthermore, U.S. Pat. No. 9,050,311 describes crystalline Forms A and B of treprostinil diethanolamine.

### V. DEUTERATED TREPROSTINIL

To eliminate foreign substances such as drugs, the animal body expresses a variety of enzymes, such as cytochrome P₄₅₀ enzymes, esterases, proteases, reductases, dehydrogenases and monoamine oxidases, which react with and convert the foreign substances to more polar intermediates or metabolites for renal excretion. Such metabolic reactions can involve, e.g., the oxidation of a carbon-hydrogen (C—H) bond to a carbon-oxygen (C—O) bond or a carbon-carbon (C═C) pi bond, or a carbon-oxygen (C—O) single bond to a carbon-oxygen (C═O) double bond. The resulting metabolites may be stable or unstable under physiological conditions, and may have substantially different pharmacologic, pharmacokinetic and pharmacodynamic properties and toxicity profiles compared to the parent compounds. For many drugs, such metabolic oxidations can be rapid and lead to the requirement of higher dosage amounts or/and increased dosing frequencies, which can result in greater side effects.

The present disclosure encompasses treprostinil isotopologues that are enriched with deuterium (deuterated) at one or more positions. In some embodiments, treprostinil or a salt thereof is deuterated at one or more positions. Deuteration of treprostinil or a salt thereof at one or more positions can have any one or more, or all, of the following benefits: (1) a longer half-life; (2) decreased amount of a dose or/and decreased number of doses needed to achieve a desired effect; (3) decreased variation between subjects in the blood or plasma level of treprostinil; (4) increased efficacy; (5) reduced side effects due to decreased amount of treprostinil administered or/and decreased production of deleterious metabolites; and (6) increased maximum tolerated dose.

Deuterium can be substituted for hydrogen at any one or more, or all, of the available positions in treprostinil (Trp) or a salt thereof, including at any one or more, or all, of the available positions in the phenyl ring of Trp, the cyclohexyl ring of Trp, the cyclopentyl ring of Trp, the octyl chain of Trp, or the hydroxyacetic acid group of Trp, or any combination thereof. In some embodiments, treprostinil or a salt thereof is deuterated at one or more, or all, of the available positions in the cyclohexyl ring of Trp, the octyl chain of Trp or the hydroxyacetic acid group of Trp, or any combination or all thereof. In certain embodiments, treprostinil or a salt thereof is any deuterated treprostinil compound disclosed in US 2011/0294815 or a salt thereof. In some embodiments, at least one of the available positions has deuterium enrichment of at least about 10%, 25%, 50%, 75%, 90%, 95% or 98% (e.g., at least about 50%). In certain embodiments, at least one of the available positions has deuterium enrichment of at least about 90%, 95% or 98%.

In further embodiments, each position in treprostinil or a salt thereof enriched with deuterium (or deuterated) independently has deuterium enrichment of at least about 10%, 25%, 50%, 75%, 90%, 95% or 98% (e.g., at least about 50%). In certain embodiments, each position enriched with deuterium independently has deuterium enrichment of at least about 90%, 95% or 98%.

The term “deuterium enrichment” refers to the percentage of incorporation of deuterium at a given position in a molecule in place of hydrogen. For example, deuterium enrichment of 10% at a given position means that 10% of molecules in a given sample contain deuterium at that position. Because the naturally occurring distribution of deuterium is about 0.0156%, deuterium enrichment at any position in a molecule synthesized using non-deuterium-enriched starting materials or reagents is about 0.0156%. Deuterium enrichment can be determined using conventional analytical methods known to one of ordinary skill in the art, including mass spectrometry and nuclear magnetic resonance spectroscopy.

The term “is deuterium” or “is deuterated”, when used to describe a given position in a molecule, or the symbol “D”, when used to represent an element at a given position in a drawing of a molecular structure, means that the specified position is enriched with deuterium above the naturally occurring distribution of deuterium. In some embodiments, deuterium enrichment is at least about 10%, 20%, 30%, 40%, 50%, 60%, 70%, 80%, 90%, 95%, 98% or 99% (e.g., at least about 50%) of deuterium at the specified position. In certain embodiments, deuterium enrichment is at least about 90%, 95% or 98% of deuterium at the specified position.

Deuterated treprostinil or a salt thereof can also contain less prevalent isotopes for other elements, including without limitation ¹³C or ¹⁴C for carbon and ¹⁷O or ¹⁸O for oxygen.

### VI. DERMAL/TRANSDERMAL ADMINISTRATION OF TREPROSTINIL

The present disclosure describes methods, compositions, devices and systems for dermal and transdermal administration of treprostinil and salts thereof, which can provide suitable local or systemic availability of treprostinil and salts thereof without causing significant or excessive skin irritation. In some embodiments, a treprostinil-containing composition, device or system is applied to the surface of the skin of a subject, from where treprostinil or a salt thereof can diffuse through the skin into the blood circulation if desired. In addition to treprostinil or a salt thereof, a dermal or transdermal pharmaceutical composition, device or system can contain one or more pharmaceutically acceptable excipients or carriers, and can optionally contain an additional therapeutic agent.

Advantages of transdermal administration can include circumvention of the gastrointestinal tract (including enzymes and acid in the GI tract and absorption through it) and hepatic first-pass metabolism; delivery of a therapeutic agent with a short half-life, a small therapeutic index or/and low oral bioavailability; controlled, continuous and sustained release of the therapeutic agent; a more uniform plasma level or delivery profile of the therapeutic agent; lower dose and less frequent dosing of the therapeutic agent; reduction of systemic side effects (e.g., side effects caused by a temporary overdose or an overly high peak plasma drug concentration); minimal or no invasiveness; ease of self-administration; and increased patient compliance.

The skin provides a strong barrier to the flux of exogenous agents into the skin. Structurally, the skin comprises two principle portions: 1) a relatively thin outer layer (the epidermis), and 2) a thicker inner region (the dermis). The outermost layer of the epidermis (the stratum corneum) contains flattened dead cells that are filled with keratin. The region between the flattened dead cells of the stratum corneum is filled with lipids that form lamellar phases. The impervious nature of the skin is due primarily to the stratum corneum, namely the intercellular multi-lamellar lipid bilayers therein. The viable epidermis underlying the stratum corneum is akin to other living tissues. The dermis provides the skin's structural strength as well as nerve and vascular networks that support the epidermis. The hypodermis, which is below the dermis, also supplies the skin with nerves and blood vessels.

Dermal administration and transdermal administration include without limitation administration onto the skin, into the skin, through the skin and across the skin. Therefore, dermal administration and transdermal administration include, but are not limited to, application to the surface of the skin as well as intradermal administration within or into a layer of the skin (e.g., epidermis or dermis) or between layers of the skin, such as by injection using a syringe and needle or a jet injector, infusion using a wearable microinfusion pump coupled with a small needle, insertion of microneedles attached to a patch, or implantation of an intradermal implant. The dermis contains a rich capillary bed for systemic drug absorption just below the epidermal-dermal junction. In some embodiments, dermal or transdermal administration of treprostinil or a salt thereof, and optionally an additional therapeutic agent, delivers the therapeutic agent(s) into the blood for systemic distribution (e.g., to treat pulmonary hypertension, such as PAH).

Delivery of a therapeutic agent into the skin in sufficient concentration often requires some means for reducing the stratum corneum's hindrance of penetration. A number of methods for reducing or circumventing the stratum corneum's barrier properties have been developed, including electrically assisted techniques such as ultrasound and electroporation, and bypassing of the stratum corneum through thermal ablation, radio frequency, microdermabrasion or microneedles. Chemical permeation/penetration enhancers can effectively and temporarily increase skin permeability by reversibly disrupting the lipid bilayers of the stratum corneum, but have the potential to irritate the cells of the viable epidermis.

A composition, device or system (e.g., a patch) can transdermally deliver treprostinil or a salt thereof, and optionally an additional therapeutic agent, passively (passive transport of treprostinil as the carboxylic acid in some embodiments) via a concentration gradient (with or without the use of a chemical permeation enhancer that can increase skin permeability or/and can increase drug partitioning/solubility in the skin and thereby increase the drug concentration gradient) or via an active mechanism (e.g., iontophoresis or microneedles). The drug(s)' absorption into and diffusion through the skin can be facilitated by the use of one or more chemical or/and physical enhancement methods that increase skin permeability or/and provide an added driving force for drug transport into and through the skin, such as one or more chemical permeation enhancers (e.g., a surfactant [e.g., sodium laureth sulfate], optionally in combination with an aromatic compound [e.g., 1-phenylpiperazine]), iontophoresis, non-cavitational ultrasound, cavitational ultrasound, electroporation, thermal ablation, radio frequency, microdermabrasion, microneedles or high pressure, or any combination thereof.

Chemical permeation enhancers can increase skin permeability by disrupting the intercellular multi-lamellar lipid bilayers in the stratum corneum, and can provide an added driving force for drug transport by increasing drug partitioning/solubility in the skin and thereby increase the drug concentration gradient driving drug diffusion through the skin. Iontophoresis typically applies a continuous low-voltage electrical current and mainly provides an electrical driving force for drug transport across the stratum corneum and the skin. Charged molecules are moved via electrophoresis, while weakly charged and uncharged molecules can be moved by convective flow of water generated by the preferential movement of mobile cations (e.g., Na⁺) instead of fixed anions (e.g., keratin) in the stratum corneum, a process called electro-osmosis. Use of a low current does not cause significant skin irritation. Compared to passive transdermal techniques, iontophoretic techniques can provide faster drug release into the skin, better control of the rate and amount of drug delivery, and greater ability to diffuse a charged molecule or a macromolecule through the skin. Ultrasound is an oscillating pressure wave at a frequency too high for humans to hear. Non-cavitational ultrasound, which generates heat but not bubbles, increases skin permeability by disrupting the stratum corneum lipid structure. Cavitational ultrasound generates heat and bubbles that oscillate and collapse at the skin surface, resulting in disruption of the stratum corneum lipid structure, which increases skin permeability for up to many hours without damaging deeper tissues. Low-frequency (kHz, such as about 55 kHz) ultrasound applied to the skin for an average duration of, e.g., about 15 seconds results in the formation and oscillation of cavitational bubbles and greater percutaneous drug flux.

Electroporation uses short (microseconds to milliseconds), high-voltage (e.g., about 100 V) electrical pulses that disrupt the lipid bilayer structures in the stratum corneum and thereby mainly enhances skin permeability. Drug diffusion through long-lived electropores can persist for up to hours. Thermal ablation increases skin permeability via formation of micron-scale perforations in the stratum corneum by heating the skin surface to hundreds of degrees for microseconds to milliseconds using a laser for example, without damaging the epidermis or deeper tissues. The pores created in the stratum corneum allow a drug applied to the surface of the skin to bypass the stratum corneum diffusional barrier and to gain access to the vascularized deeper layers of the skin. Radio frequency-based permeation exposes the skin to high-frequency alternating current for, e.g., less than about one second, which forms heat-induced microchannels in the skin. The rate of drug delivery is controlled by the number and depth of microchannels. Microdermabrasion enhances skin permeability through removal of the stratum corneum barrier by, e.g., blowing microcrystals onto the skin surface. Insertion of microneedles (very short needles with a length of, e.g., about 50-110 microns, such as about 100 microns) penetrates the stratum corneum and enhances skin permeability by creating micron-scale channels into the skin. Microneedles can actively drive a drug into the skin via coating of solid microneedles with the drug, encapsulation of the drug within bioabsorbable polymeric microneedles, or convective flux of the drug through hollow microneedles. Alternatively, after the skin is pierced with microneedles to form microchannels into the skin, a needleless patch can be applied to the pierced skin to deliver the drug into the skin via the microchannels. Jet injection uses a high-pressure narrow jet of injection liquid to penetrate the epidermis and thereby creates micron-scale holes in the skin. A jet injector forces compressed air or gas (e.g., helium) through a nozzle and shoots drug particles entrained in the jet flow at high velocity for skin penetration. A device (e.g., a patch or a jet injector) containing a drug reservoir or depot can be designed so that high pressure applied to the device, or high pressure generated by the device, causes the drug to be directed out of the device (optionally through a small needle or microneedles) and through the stratum corneum. Application of modest pressure (e.g., about 25 kPa) may also suffice for skin penetration of a drug.

Combinations of chemical or/and physical enhancement techniques are often more effective in increasing transdermal drug transport than the individual techniques alone, and can do so in a synergistic manner. A combination of chemical or/and physical enhancers can also reduce the required “dose” of each enhancer. Such combinations of enhancement techniques can include, but are not limited to, chemical penetration enhancer(s) (CPE(s), such as limonene, oleic acid, Azone, ethanol or dimethyl sulfoxide, or any combination thereof) and iontophoresis; CPE(s) (e.g., linoleic acid, isopropyl myristate, a surfactant [e.g., sodium lauryl sulfate] or polyethylene glycol, or any combination thereof) and ultrasound; CPE(s) (e.g., a polysaccharide [e.g., dextran or heparin], urea or sodium thiosulfate, or any combination thereof) and electroporation; iontophoresis and ultrasound; iontophoresis and electroporation; and ultrasound and electroporation.

In some embodiments, treprostinil or a salt thereof, and optionally an additional therapeutic agent, are administered through the use of an enhancement technique that increases skin permeability in combination with an enhancement technique that provides an additional driving force for drug transport. In some embodiments, iontophoresis, which mainly provides a driving force for drug transport, is employed in combination with one or more chemical or/and physical enhancement techniques that increase skin permeability. Examples of such a combination include without limitation iontophoresis-chemical permeation enhancer, iontophoresis-ultrasound, iontophoresis-electroporation, iontophoresis-thermal ablation, iontophoresis-radio frequency, iontophoresis-microdermabrasion, iontophoresis-microneedles, and iontophoresis-high pressure.

Chemical or/and physical enhancement methods (e.g., chemical permeation enhancers, iontophoresis, cavitational ultrasound and microneedles) can be integrated into or with a transdermal drug-delivery device or system, such as a patch. For instance, ultrasound can be applied using a hand-held device, or a low-frequency, cymbal transducer that can be integrated into a sonophoretic patch (see, e.g., U.S. Pat. No. 9,327,105). The rate of drug delivery can be controlled, e.g., by a semi-permeable membrane or a polymer matrix of a patch; by real-time skin impedance feedback that stops a sonication procedure when the desired level of conductance has been achieved; or by iontophoresis as a function of the electrical current, which can be controlled by a microprocessor or by the patient and permits continuous or intermittent drug delivery. As another example, an electronically controlled micropump can be integrated into or with a patch containing solid or hollow microneedles to control the timing, frequency and length of drug delivery. As an additional example, an iontophoretic patch can contain a battery to allow for on-demand drug delivery, which permits a quicker onset of drug action.

A physical enhancement method can be applied using, e.g., a hand-held, electrically powered device that interfaces with a disposable component containing a drug reservoir. For example, a thermal ablation system can comprise a reusable hand-held applicator and a disposable drug-containing patch.

In some embodiments, passive transdermal administration of treprostinil, with or without the use of one or more chemical permeation enhancers, delivers treprostinil as the free carboxylic acid. In other embodiments, a salt form of treprostinil (e.g., an alkali metal salt such as treprostinil sodium or an amine salt such as treprostinil diethanolamine) is transdermally delivered with the assistance of iontophoresis.

For a delayed or sustained release over a period of at least about 1 week, 2 weeks, 1 month, 2 months, 3 months, 6 months, 1 year or longer, treprostinil or a salt thereof can be delivered via a variety of devices, such as an intradermal or transdermal implant or an extended-release transdermal or microneedles patch.

Pharmaceutically acceptable excipients and carriers include pharmaceutically acceptable substances, materials and vehicles. Non-limiting examples of excipients include liquid and solid fillers, diluents, binders, lubricants, glidants, surfactants, dispersing agents, disintegration agents, emulsifying agents, wetting agents, suspending agents, thickeners, solvents, isotonic agents, buffers, pH adjusters, absorption-delaying agents, stabilizers, preservatives, antioxidants, antimicrobial agents, antibacterial agents, antifungal agents, adjuvants, encapsulating materials and coating materials. The use of such excipients in pharmaceutical formulations is known in the art. For example, conventional vehicles and carriers include without limitation oils (e.g., vegetable oils, such as sesame oil), aqueous solvents (e.g., saline, phosphate-buffered saline [PBS] and isotonic solutions [e.g., Ringer's solution]), and solvents (e.g., dimethyl sulfoxide [DMSO] and alcohols [e.g, ethanol, glycerol and propylene glycol]). Except insofar as any conventional excipient or carrier is incompatible with the active ingredient, the disclosure encompasses the use of conventional excipients and carriers in formulations containing treprostinil or a salt thereof. See, e.g., Remington: The Science and Practice of Pharmacy, 21st Ed., Lippincott Williams & Wilkins (Philadelphia, Pa. [2005]); Handbook of Pharmaceutical Excipients, 5th Ed., Rowe et al., Eds., The Pharmaceutical Press and the American Pharmaceutical Association (2005); Handbook of Pharmaceutical Additives, 3rd Ed., Ash and Ash, Eds., Gower Publishing Co. (2007); and Pharmaceutical Pre-formulation and Formulation, Gibson, Ed., CRC Press LLC (Boca Raton, Fla. [2004]).

The disclosure includes kits comprising a treprostinil-containing composition, device or system or a plurality thereof, wherein all of the necessary components of the composition, device or system can be provided as a complete unit (e.g., a transdermal patch) or can be provided separately for assemblage. A transdermal patch, or a plurality thereof, can be stored in any suitable container, such as a pouch (e.g., a resealable pouch). The kits can further comprise any instrument(s) or device(s) required for the application of a physical enhancement technique if desired, or such instrument(s) or device(s) can be part of the treprostinil-containing composition, device or system (e.g., an iontophoretic patch or a sonophoretic patch). The kits can contain instructions for storing, preparing (if necessary), and applying or using the treprostinil-containing composition, device or system and any other instrument(s) or device(s). The instructions for applying or using the treprostinil-containing composition, device or system and any other instrument(s) or device(s) can be tailored to the treatment of a particular medical condition, such as pulmonary hypertension or more specifically PAH.

A. Dermal/Transdermal Compositions

Topical formulations for application to the skin can be useful for treating conditions of the upper skin layers, or for transdermal administration of a therapeutic agent (e.g., treprostinil or a salt thereof) to the local tissue underlying the skin (which can be useful for treating, e.g., ischemic ulcers such as digital ulcers) or into the blood for systemic distribution. In general and in addition to the disclosure on dermal/transdermal compositions described elsewhere herein, topical compositions suitable for application to the skin include without limitation oils, liquid or semi-liquid preparations such as sprays, gels, jellies, liniments, lotions, and oil-in-water or water-in-oil emulsions such as creams, foams, ointments and pastes. Application of a spray, a gel or other topical formulation to the skin, upon evaporation or absorption, can drive a lipophilic drug of low molecular weight into the stratum corneum (the skin's outermost layer), which can serve as a drug reservoir for extended release of the drug into the viable epidermis (the skin's middle layer) over a period of hours. A topical composition can also be configured as a dressing impregnated with a therapeutic agent and composed of, e.g., a gel, a foam, a paste, a hydrogel, a hydrocolloid, a hydrocellular material or a hydrofiber, or any combination thereof.

In some embodiments, a topical composition comprises treprostinil or a salt thereof, and optionally an additional therapeutic agent, dissolved, suspended, dispersed or incorporated in a carrier. The carrier can be in the form of, e.g., a solution, a suspension, an emulsion, an ointment, a gel base, a liquid or gel composition of a patch, or a polymer matrix of a patch, and can contain, e.g., petrolatum, lanolin, a wax (e.g., bee wax), mineral oil, a long-chain alcohol, polyethylene glycol or polypropylene glycol, a diluent (e.g., water or/and an alcohol [e.g., ethanol or propylene glycol]), a gel, a polymeric material, an emulsifier, a thickening agent, a stabilizer or a preservative, or any combination thereof. In certain embodiments, the carrier comprises the drug in a concentration of about 0.1-15% w/w, 0.1-10% w/w, 0.5-5% w/w or 0.5-2% w/w.

In some embodiments, a topical composition comprises a chemical penetration/permeation enhancer (CPE) that facilitates transport of treprostinil or a salt thereof (e.g., passive transport of treprostinil as the free carboxylic acid), and optionally an additional therapeutic agent, into the skin or/and across the skin into systemic circulation. Non-limiting examples of CPEs include hydrocarbons (e.g., alkanes and alkenes [e.g., squalene]); terpenes and terpenoids (e.g., D-limonene, carvone, eucalyptol, menthol, menthone and nerolidol); essential/volatile oils (e.g., anise oil, caraway oil, cardamom oil, chenopodium oil, eucalyptus oil and lemon oil); ethers and fatty ethers (e.g., 2-n-nonyl-1,3-dioxolane); phenols (e.g., eugenol); alcohols and fatty alcohols (e.g., methanol, ethanol, isopropyl alcohol, pentanol, lauryl alcohol, oleyl alcohol, benzyl alcohol, propylene glycol, dipropylene glycol, polyethylene glycol and glycerol); benzoic acids (e.g., salicylic acid and acetylsalicylic acid); fatty acids (e.g., valeric acid, lauric acid, oleic acid and linoleic acid); esters, fatty alcohol esters and fatty acid esters (e.g., ethyl acetate, methyl laurate, isopropyl myristate, isopropyl palmitate, methyl oleate, ethyl oleate, propylene glycol mono-oleate, glycerol mono-oleate, triacetin and pentadecalactone); hydroxyl-containing esters, fatty alcohol esters and fatty acid esters (e.g., lauryl lactate, glyceryl/glycerol monolaurate, glycerol monoleate [mono-olein], sorbitan oleate and octyl salicylate); amines (e.g., diethanolamine and triethanolamine); amides, fatty amine amides and fatty acid amides (e.g., urea, dimethylformamide, dimethylacetamide, diethylacetamide, diethyltoluamide, N-lauroyl sarcosine, 1-dodecylazacycloheptane-2-one [laurocapram or Azone®], Azone-related compounds, and pyrrolidone compounds [e.g., 2-pyrrolidone and N-methyl-2-pyrrolidone]); ionic and non-ionic surfactants (e.g., cetyltrimethylammonium bromide, sodium laurate, sodium laureth sulfate [sodium lauryl ether sulfate], sodium cholate, sorbitan monolaurate, Brij® surfactants, Pluronic® surfactants, and Tween® surfactants); phospholipids (e.g., lecithin); sulfoxides (e.g., dimethyl sulfoxide); ginsenosides and those described elsewhere herein. US Pub. 2007/0269379 provides an extensive list of CPEs.

In certain embodiments, the CPE includes a surfactant. In some embodiments, the CPE includes two or more surfactants, such as a non-ionic surfactant (e.g., sorbitan monolaurate or N-lauroyl sarcosine) and an ionic surfactant (e.g., an anionic surfactant, such as sodium lauroyl sarcosinate). In further embodiments, the CPE includes a surfactant (e.g., an anionic surfactant, such as sodium laureth sulfate [sodium lauryl ether sulfate]) and an aromatic compound (e.g., 1-phenylpiperazine). Such combinations of CPEs can greatly enhance permeation of drug(s) through the skin with a low skin irritation potential. In additional embodiments, the CPE includes an organic sulfoxide and a compound selected from fatty acids, fatty acid esters and Azone-related compounds.

Heat can also enhance skin penetration. Heat can be in the form of, e.g., radiating heat, conductive heat or convective heat. Radiating heat can be provided by, e.g., an infrared lamp.

A topical composition can be part of, comprise, or be integrated into or with a transdermal delivery device or system (e.g., a patch). Furthermore, a topical composition can be applied in combination with any of the physical enhancement techniques described herein (e.g., iontophoresis).

Additional examples of dermal/transdermal compositions are described below for purposes of illustration.

1. Compositions Comprising a Permeation Enhancer

In some embodiments, a topical composition comprises treprostinil or a salt thereof and a permeation enhancer. The composition can optionally contain an additional therapeutic agent.

The permeation enhancer increases the permeability of the skin to the therapeutic agent(s). In certain embodiments, the permeation enhancer includes N-lauroyl sarcosine, sodium octyl sulfate, methyl laurate, isopropyl myristate, oleic acid, glyceryl oleate or sodium lauryl sulfoacetate, or any combination thereof. In certain embodiments, the composition contains on a weight/volume (w/v) basis the permeation enhancer in an amount of about 1-20%, 1-15%, 1-10% or 1-5%. To enhance further the ability of the therapeutic agent(s) to penetrate the skin, the composition can also contain a surfactant, Azone® or an Azone-like compound, an alcohol, a fatty acid, a fatty ester, or an aliphatic thiol, or any combination thereof.

The composition can further contain one or more additional excipients. Suitable excipients include without limitation solubilizers (e.g., C₂-C₈ alcohols), moisturizers or humectants (e.g., glycerol [glycerin], propylene glycol, amino acids and derivatives thereof, polyamino acids and derivatives thereof, and pyrrolidone carboxylic acids and salts and derivatives thereof), surfactants (e.g., sodium laureth sulfate and sorbitan monolaurate), emulsifiers (e.g., cetyl alcohol and stearyl alcohol), thickeners (e.g., methyl cellulose, ethyl cellulose, hydroxymethyl cellulose, hydroxypropyl cellulose, polyvinylpyrrolidone, polyvinyl alcohol and acrylic polymers), and formulation bases or carriers (e.g., polyethylene glycol as an ointment base). As a non-limiting example, the base or carrier of the composition can contain ethanol, propylene glycol and polyethylene glycol (e.g., PEG 300), and optionally an aqueous liquid (e.g., isotonic phosphate-buffered saline).

The topical composition can have any suitable dosage form, such as an emulsion, a cream, a lotion, a gel, an ointment, a paste, a jelly, a foam or a spray. In some embodiments, the composition is applied to the skin covering a surface area of about 10-800 cm², 10-400 cm² or 10-200 cm². The composition can deliver the therapeutic agent(s) to the skin or the underlying tissue. The composition can also be formulated for transdermal administration of the therapeutic agent(s) to the systemic circulation as, e.g., a transdermal patch or a microneedles patch.

2. Compositions Comprising a Permeation Enhancer and a Volatile Liquid

In further embodiments, a topical composition comprises treprostinil or a salt thereof, a permeation enhancer and a volatile liquid. The composition can optionally contain an additional therapeutic agent.

The permeation enhancer increases the permeability of the skin to the therapeutic agent(s). In some embodiments, the permeation enhancer is selected from C₅-C₁₈ alkyl aminobenzoates (e.g., C₈-C₁₈ alkyl p-aminobenzoates), C₅-C₁₈ alkyl dimethylaminobenzoates (e.g., C₈-C₁₈ alkyl p-dimethylaminobenzoates), C₅-C₁₈ alkyl cinnamates, C₅-C₁₈ alkyl methoxycinnamates (e.g., C₈-C₁₈ alkyl p-methoxycinnamates), and C₈-C₁₈ alkyl salicylates. In certain embodiments, the permeation enhancer includes octyl salicylate, octyl p-dimethylaminobenzoate or octyl p-methoxycinnamate, or any combination thereof.

The volatile liquid can be any volatile, skin-tolerant solvent. In certain embodiments, the volatile liquid is a C₂-C₅ alcohol or an aqueous solution thereof, such as ethanol or isopropanol or an aqueous solution thereof. An aerosol propellant (e.g., dimethyl ether) can be considered as a volatile liquid. In some embodiments, the volatile liquid functions as a carrier or vehicle of the composition.

The composition can optionally contain a thickening agent. Non-limiting examples of thickening agents include cellulosic thickening agents (e.g., ethyl cellulose, hydroxypropyl cellulose and hydroxypropyl methylcellulose), povidone, polyacrylic acids/polyacrylates (e.g., Carbopol® polymers), Sepigel® (polyacrylamide/isoparaffin/laureth-7), and the Gantrez® series of polymethyl vinyl ether/maleic anhydride copolymers (e.g., butyl ester of PMV/MA copolymer Gantrez® A-425).

In some embodiments, the composition contains on a weight basis about 0.1-5%, 0.5-5% or 1-5% of treprostinil or a salt thereof, about 1-20%, 1-15% or 1-10% of the permeation enhancer, and about 40-98%, 45-95%, 50-90% or 60-80% of the volatile liquid. In further embodiments, the composition optionally contains on a weight basis about 1-40%, 1-30%, 1-20% or 5-20% water or/and about 0.1-15%, 0.5-10% or 1-5% of a thickening agent.

For purposes of illustration, in certain embodiments a topical spray composition contains about 0.1-5% w/v of treprostinil or a salt thereof, about 2-10% w/v of octyl salicylate or octyl p-methyoxycinnamate, and about 95% aqueous ethanol as the carrier. In further embodiments, a topical gel composition comprises about 0.1-5% w/v of treprostinil or a salt thereof, about 1-10% w/v of octyl salicylate or octyl p-methyoxycinnamate, about 0.5-5% w/v of a Carbopol® polyacrylic acid, and about 70% aqueous ethanol as the carrier, and optionally about 1-10% w/v of a basic solution (e.g., 0.1 N NaOH). In additional embodiments, a topical lotion composition contains about 0.1-5% w/v of treprostinil or a salt thereof, about 1-10% w/v of octyl salicylate or octyl p-methyoxycinnamate, about 1-5% w/v of ethyl cellulose or hydroxypropyl cellulose, and about 90% aqueous ethanol as the carrier.

The composition can further comprise other excipients, such as a compounding agent (e.g., paraffin oil, silicone oil, a vegetable oil, or a fatty ester such as isopropyl myristate), a diluent, a co-solvent (e.g., acetone or a glycol ether such as diethylene glycol monoethyl ether), an emulsifier, a surfactant (e.g., an ethoxylated fatty alcohol, glycerol mono stearate or a phosphate ester), a stabiliser, an antioxidant or a preservative (e.g., a hydroxybenzoate ester), or any combination thereof. For example, a co-solvent or/and a surfactant can be used to maintain the therapeutic agent(s) in solution or suspension at the desired concentration.

The topical composition can have any suitable dosage form, such as a cream, a lotion, a gel, an ointment, a mousse, a spray or aerosol, or any transdermal device (e.g., a patch) that administers a drug by absorption through the skin. In some embodiments, the topical composition is applied to the skin covering a surface area of about 10-800 cm², 10-400 cm² or 10-200 cm².

3. Compositions Including a Permeation Enhancer and Another Excipient

In additional embodiments, a topical composition comprises treprostinil or a salt thereof, a permeation enhancer, and at least one of a lipophilic solvent, a formulation base and a thickener. In some embodiments, the composition contains a lipophilic solvent and a formulation base, or the same substance can function as both a lipophilic solvent and a formulation base. In further embodiments, the composition contains a lipophilic solvent, a formulation base and a thickener. The composition can optionally comprise an additional therapeutic agent.

The permeation enhancer increases the permeability of the skin to the therapeutic agent(s). Non-limiting examples of permeation enhancers include dimethyl sulfoxide (DMSO), decylmethylsulfoxide, laurocapram, pyrrolidones (e.g., 2-pyrrolidone and N-methyl-2-pyrrolidine), surfactants, alcohols (e.g., oleyl alcohol), polyethylene glycol (e.g., PEG 400), diethylene glycol monoethyl ether, oleic acid, and fatty acid esters (e.g., isopropyl myristate, methyl laurate, glycerol monooleate, and propylene glycol monooleate).

Non-limiting examples of lipophilic solvents include lipophilic alcohols (e.g., hexylene glycol, octyldodecanol, oleyl alcohol and stearyl alcohol), polyethylene glycol (e.g., PEG 100, PEG 300, PEG 400 and PEG 3350), diethylene glycol monoethyl ether, polysorbates (e.g., Tween® 20 to 80), Labrasol®, fatty acid esters (e.g., isopropyl myristate and diisopropyl adipate), diethyl sebacate, propylene glycol monocaprylate, propylene glycol laurate, mono- and di-glycerides (e.g., Capmul® MCM), medium-chain triglycerides, caprylic/capric triglyceride, glyceryl monocaprylate, glyceryl mono-oleate, glyceryl mono-linoleate, glycerol oleate/propylene glycol, mineral oil, and vegetable oils.

A lipophilic solvent may also function as a formulation base or carrier. For example, polyethylene glycol (e.g., from PEG 100 to PEG 3500, such as PEG 300, PEG 400 and PEG 3350) can function as a lipophilic solvent and a formulation base.

The composition can also contain a hydrophilic solvent, such as a C₁-C₅ alcohol (e.g., ethanol, isopropanol, glycerol, propylene glycol and 1,2-pentanediol) or/and water.

The composition can contain a thickener to increase the viscosity or/and the physical stability of the composition. Examples of thickeners include without limitation glycerol, stearyl alcohol, and polymers (e.g., polydimethylsiloxane [dimethicone] and Carbopol® polymers).

In some embodiments, the composition further contains an antioxidant. Non-limiting examples of antioxidants include butylated hydroxyanisole (BHA), butylated hydroxytoluene (BHT), tocopherols (e.g., vitamin E and esters thereof), flavonoids, glutathione, ascorbic acid and esters thereof, DMSO, and chelating agents (e.g., EDTA and citric acid).

In certain embodiments, the topical composition comprises on a w/w basis about 0.1-5% or 0.5-5% of treprostinil or a salt thereof, about 2-30% or 5-20% of a permeation enhancer, about 20-80% or 30-70% of a lipophilic solvent that may also function as a formulation base, about 0.1-10% or 1-7.5% of a thickener, and about 0.01-2% or 0.05-1% of an antioxidant. As a non-limiting example, a topical composition can contain treprostinil or a salt thereof, PEG 400 or/and PEG 3350 as lipophilic solvent(s) and formulation base(s), diethylene glycol monoethyl ether, oleyl alcohol or/and isopropyl myristate as permeation enhancer(s), stearyl alcohol as a thickener, and BHT as an antioxidant.

The topical composition can have any suitable dosage form, such as a cream, a lotion, a gel, an ointment, a jelly, a paste, or any transdermal device (e.g., a patch) that administers a drug by absorption through the skin.

B. Transdermal Drug-Delivery Devices/Systems

In some embodiments, treprostinil or a salt thereof, and optionally an additional therapeutic agent, are administered by means of a transdermal drug-delivery device or system (TDS), such as a transdermal patch, a microneedles patch or an iontophoresis device (e.g., an iontophoretic patch). Advantages of a TDS can include controlled, prolonged, continuous delivery of a therapeutically effective amount of one or more drugs into the systemic circulation at a substantially constant rate (substantially zero-order kinetics), a more uniform concentration of the drug(s) in the blood, ease of use and increased patient compliance. The TDS can transdermally deliver the drug(s) passively (with or without the use of a chemical permeation enhancer) or actively (using, e.g., iontophoresis or microneedles). The drug(s)′ absorption into and diffusion through the skin can be facilitated by the use of one or more chemical or/and physical enhancement methods that increase skin permeability or/and provide an added driving force for drug transport into and through the skin, such as one or more chemical permeation enhancers (e.g., a surfactant [e.g., sodium laureth sulfate], optionally in combination with an aromatic compound [e.g., 1-phenylpiperazine]), iontophoresis, non-cavitational ultrasound, cavitational ultrasound, electroporation, thermal ablation, radio frequency, microdermabrasion, microneedles or high pressure, or any combination thereof. Such enhancement methods (e.g., chemical permeation enhancers, iontophoresis, cavitational ultrasound and microneedles) can be integrated into or with a TDS such as a patch.

In some embodiments, treprostinil or a salt thereof, and optionally an additional therapeutic agent, are administered via a transdermal patch. A transdermal patch can contain, e.g., a drug reservoir that is enclosed on one side with an impermeable backing layer/film and has a skin-contacting adhesive layer on the other side. The backing layer protects the patch and its contents from the outer environment and can be composed of any suitable material (e.g., a metallic plastic laminate). The patch can have a liner that protects the patch during storage and is removed immediately prior to patch application. A therapeutic agent can be dissolved, suspended or dispersed in a liquid- or gel-based reservoir, which allows the use of a liquid chemical permeation enhancer (e.g., ethanol). The gel can be composed of, e.g., a suitable polymeric material (e.g., hydroxypropyl cellulose). In some embodiments, a transdermal patch comprises an impermeable backing layer, a liquid- or gel-based drug reservoir, a semi-permeable membrane that can serve as a rate-limiting or rate-controlling diffusion barrier, and a skin-contacting adhesive layer. The semi-permeable membrane can be composed of, e.g., a suitable polymeric material (e.g., cellulose nitrate or acetate, polyisobutene, polypropylene, polyethylene, polyvinyl acetate or a polycarbonate). In some embodiments where the transdermal patch is an iontophoretic patch, the patch contains an optionally buffered aqueous solution comprising the therapeutic agent and optionally an alcohol (e.g., ethanol) that can aid in dissolution of a hydrophobic therapeutic agent and can function as a chemical permeation enhancer.

Alternatively, a transdermal patch can have a therapeutic agent incorporated or dispersed in a solid or semi-solid polymer matrix. The polymer matrix (e.g., ethylene-vinyl acetate) may control the release of the therapeutic agent by controlling dissolution or/and diffusion of the agent in or from the polymer matrix, and can enhance the stability of the agent while incorporated in the matrix. In certain embodiments, a transdermal patch comprises an impermeable backing layer/film, a drug/polymer matrix, and a skin-contacting adhesive layer.

In other embodiments, a transdermal patch is a drug-in-adhesive patch, which can be regarded as a matrix patch. In some embodiments, a transdermal patch comprises an impermeable backing layer/film and a skin-contacting adhesive layer incorporating a therapeutic agent in a polymeric or viscous adhesive (a single-layer drug-in-adhesive patch). The patch can be designed so that body heat (about 37° C.) induces liquefaction of the material in the adhesive layer, dissolution of the therapeutic agent embedded in the adhesive layer and diffusion of the therapeutic agent into the skin. In further embodiments, a transdermal patch comprises an impermeable backing layer/film, an upper adhesive layer containing a therapeutic agent, a semi-permeable membrane, and a skin-contacting adhesive layer containing the therapeutic agent (a multi-layer drug-in-adhesive patch). The skin-contacting adhesive layer is designed for immediate or initial release of the therapeutic agent while the second (upper) adhesive layer is designed for controlled release of the therapeutic agent through the semi-permeable membrane.

In addition to one or more therapeutic agents, a transdermal patch can contain one or more chemical permeation enhancers (CPE(s), such as oleic acid or an ester thereof) and optionally other ingredient(s), such as a stabilizer (e.g., an antioxidant) and a preservative. Furthermore, a transdermal patch can contain one or more adjuvants that aid solubilization of the drug in the patch formulation, increase drug solubility in the skin or increase drug diffusivity through the skin, or any combination thereof, such as ethanol, dipropylene glycol, oleic acid or an ester thereof (e.g., oleyl oleate), or triacetin, or any combination thereof.

A transdermal patch can be made with heat-sealable components. Alternatively, the components of a transdermal patch can be adhered to one another using an adhesive.

A microneedles device (e.g., patch) containing very short needles that pierce the stratum corneum can also be used to deliver treprostinil or a salt thereof, and optionally an additional therapeutic agent, in a minimally invasive manner. The microneedles are very short (e.g., about 10-200 microns, such as about 100 microns, in length) and very narrow (e.g., about 10-50 microns in width). Depending on the length of the microneedles, microneedles can be designed to pierce through the stratum corneum and into the epidermis, across the epidermis or/and into the superficial dermis. Rapid or controlled release of a drug into the skin can be achieved by insertion of solid microneedles coated with the drug (e.g., by dip coating), insertion of solid microneedles made of a water-soluble polymer that encapsulates the drug within the needle matrix and is capable of dissolving in the skin, or insertion of solid microneedles that form microchannels into the skin followed by application of a transdermal patch delivering the drug. Alternatively, hollow microneedles can deliver a drug into the skin by injection or infusion. Such a patch can be integrated with microfluidics, can contain a liquid-based drug reservoir, and can contain a chip that controls administration of the drug.

TDSs, including patches, can be designed to provide controlled and prolonged release of a drug up to, e.g., about 1 week or longer. WO 1993/003696 and U.S. Pat. Nos. 3,598,122; 4,144,317; 4,201,211; 4,262,003 and 4,379,454 describe various TDSs, including patches, which can deliver a controlled amount of a drug for an extended period of time ranging from several hours to several days. Such systems can be adapted for transdermal delivery of treprostinil or a salt thereof, and optionally an additional therapeutic agent.

A TDS (e.g., a transdermal patch) can be applied onto the skin of a subject at any location appropriate for treatment of the medical condition being treated. Non-limiting examples of bodily locations for application of a TDS include the scalp, the forehead, the neck, the chest, the back (e.g., upper and lower back), the axilla, the abdomen, the buttock, the scrotum, the upper arm (e.g., lateral upper arm), the forearm (e.g., ventral and dorsal forearm), the hand (e.g., palmar and dorsal hand), the thigh (e.g., ventral, dorsal and lateral thigh), the calf, the ankle (e.g., lateral ankle), and the foot (e.g., dorsal and lateral foot and plantar foot arch). The rate of transdermal drug delivery may vary between bodily locations due in part to varying thickness of the stratum corneum. In some embodiments, for transdermal drug delivery into the bloodstream to treat, e.g., pulmonary hypertension (such as PAH), a TDS (e.g., a transdermal patch) is applied onto the lower back, the chest, the thigh, the buttock, the abdomen, the upper arm or the axilla of the subject. For more secure attachment of a patch to the skin, the site of patch application is preferably hairless. To prevent or reduce any side effect (e.g., skin irritation) that may be caused by a TDS, the sites of application of new or fresh TDSs (e.g., transdermal patches) can be rotated or be new ones at appropriate location(s) on the body.

Additional examples of transdermal drug-delivery devices and systems are described below for purposes of illustration.

1. TDSs Comprising a Permeation Enhancer

In some embodiments, a TDS comprises treprostinil or a salt thereof and a permeation enhancer. The TDS can optionally contain an additional therapeutic agent.

The permeation enhancer increases the permeability of the skin to the therapeutic agent(s). The permeation enhancer can be, e.g., a fatty acid ester having a fatty acyl chain length of C₈-C₂₀ or C₁₂-C₁₈ and a C₁-C₆ or C₂-C₄ alcohol component (e.g., isopropanol). In certain embodiments, the permeation enhancer includes isopropyl myristate or isopropyl palmitate. In some embodiments, the permeation enhancer is in an amount of about 0.1-20%, 0.5-15%, 1-15%, 2-12% or 4-10% by weight of the TDS (e.g., a transdermal patch) or a portion thereof (e.g., the drug reservoir or the skin-contacting layer).

In some embodiments, the TDS (e.g., a transdermal patch) comprises an adhesive that maintains contact of the TDS to the skin. Non-limiting examples of adhesives include acrylics/acrylates (e.g., polyacrylates, including polyalkyl acrylates and Duro-Tak® polyacrylates), polyvinyl acetate, ethylene-vinyl acetate copolymers, polysiloxanes, polyurethanes, plasticized polyether block amide copolymers, natural and synthetic rubbers, plasticized styrene-butadiene rubber block copolymers (e.g., Duro-Tak® 87-6173), and mixtures thereof.

The TDS can comprise one or more additional excipients. The additional excipient(s) can include, e.g., a diluent, an emollient, a plasticizer, or an agent that reduces irritation to the skin, or any combination thereof.

In certain embodiments, the TDS prior to application to the skin is substantially free of water, tetraglycol (glycofurol) or/and a hydrophilic organic solvent (e.g., a C₁-C₅ alcohol).

The TDS can administer the therapeutic agent(s) transdermally through intact unbroken skin into the systemic circulation.

In some embodiments, the TDS is in the form of a transdermal patch for application to the skin. In certain embodiments, the patch has a skin-contacting layer laminated or otherwise attached to a support layer. The skin-contacting layer can be covered by a removable release liner before use to protect the skin-contacting surface and to keep it clean until it is applied to the skin. The support layer of the patch acts as a support for the skin-contacting layer and as a barrier that prevents loss of the therapeutic agent(s) in the skin-contacting layer to the environment. The material of the support layer is compatible with the therapeutic agent(s), the permeation enhancer and the adhesive, and is minimally permeable to the components of the patch. The support layer can be opaque to protect the components of the patch from degradation via exposure to ultraviolet light. The support layer is also capable of binding to and supporting the adhesive layer, yet is sufficiently pliable to accommodate the movements of the subject using the patch. The material of the support layer can be, e.g., a metal foil, a metalized polyfoil, or a composite foil or film containing a polymer (e.g., a polyester [such as polyester terephthalate] or aluminized polyester, polyethylene, polypropylene, polytetrafluoroethylene, a polyethylene-methyl methacrylate block copolymer, a polyether block amide copolymer, a polyurethane, polyvinylidene chloride, nylon, a silicone elastomer, rubber-based polyisobutylene, styrene, or a styrene-butadiene or styrene-isoprene copolymer). The release liner can be made of the same material as the support layer, or can be a film coated with an appropriate release surface.

2. Reservoir-Type Transdermal Patches

In further embodiments, treprostinil or a salt thereof, and optionally an additional therapeutic agent, are delivered from a reservoir-type transdermal patch (RTP). In some embodiments, an RTP comprises an impermeable backing layer/film, a liquid- or gel-based drug reservoir, a semi-permeable or microporous membrane that controls drug release, and a skin-contacting adhesive layer. RTPs generally have a protective release liner that covers the skin-contacting side of the patch and is removed immediately prior to use. The drug reservoir can optionally contain one or more chemical permeation enhancers (CPEs). The rate of drug release from the reservoir can be controlled by, e.g., the polymer composition, the pore size and the thickness of the semi-permeable membrane, as well as the solubility of the drug in the liquid- or gel-based material of the reservoir. The skin-contacting adhesive layer can be peripheral and not overlap or cover the semi-permeable membrane, or can overlap or cover the semi-permeable membrane. If the adhesive layer overlaps or covers the semi-permeable membrane, the drug(s) in the reservoir can equilibrate with the adhesive layer during storage so that upon application of the patch to the skin, the drug(s) in the adhesive layer can act as a priming dose of the drug(s) that saturates the skin-binding site. To prevent detachment of the patch from the skin, a separate, protective adhesive layer (e.g., a medical/surgical tape or a PatchProtect™ or Tegaderm™ dressing) can optionally be applied over the entire patch and beyond the edges of the patch. The amount of drug delivered from an RTP can depend on, e.g., the amount of drug in the drug reservoir, the permeability of the membrane, and the area of the patch applied to the skin.

A variation of an RTP has no semi-permeable or microporous membrane covering the liquid- or gel-based drug reservoir, but rather has another type of release-limiting (e.g., rate-controlling) layer (e.g., a polymeric layer) or a skin-contacting adhesive layer covering the drug reservoir. To prevent leakage of the content of a liquid-based drug reservoir, the drug reservoir can be composed of a viscous liquid (e.g., ethylene glycol or/and a silicone fluid), or/and can contain a viscosity-enhancing agent (e.g., a gum or a natural or semi-synthetic cellulose).

In some embodiments, the drug reservoir contains a gel composed of a polymer (e.g., hydroxypropyl cellulose), in which one or more drugs can be homogeneously dispersed. In other embodiments, the drug reservoir contains a viscous liquid (e.g., ethylene glycol or/and a silicone fluid), in which one or more drugs can be dissolved or suspended, or/and contains a viscosity-enhancing agent. In certain embodiments, the semi-permeable or microporous membrane is composed of a polymer (e.g., polyethylene). U.S. Pat. No. 9,289,397 discloses a list of polymers of which semi-permeable membranes can be composed. In some embodiments, the impermeable backing layer is a metallic plastic laminate or other flexible polymer (e.g., polyethylene). In further embodiments, the backing layer is occlusive, which promotes hydration of the outer layer of the skin. Hydration can enhance penetration of the drug(s) into the skin. In certain embodiments, the backing layer is composed of a metalized polymer (e.g., polyester/ethylene-vinyl acetate), the drug-reservoir gel is composed of a polymer (e.g., hydroxypropyl cellulose) and contains a CPE (e.g., isopropyl myristate), an alcohol (e.g., ethanol) that may aid solubilization of the drug(s) and may also function as a CPE, and one or more drugs, and the semi-permeable membrane is a microporous membrane composed of a polymer (e.g., polyethylene).

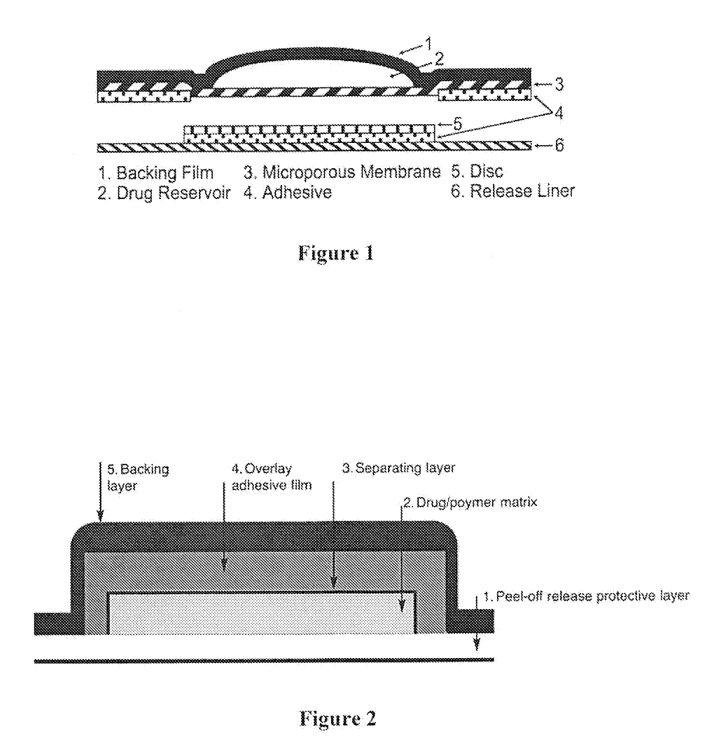

In certain embodiments, an RTP has the structure depicted in FIG. 1. The RTP of FIG. 1 has the following layers:

(1) Layer 1 is a backing layer or film composed of, e.g., a metalized polymer (e.g., polyester/ethylene-vinyl acetate) and optionally a material impermeable to light (e.g., an ink);

(2) Layer 2 is a drug-reservoir gel composed of, e.g., a polymer (e.g., hydroxypropyl cellulose) and containing one or more drugs and optionally a CPE (e.g., isopropyl myristate) or/and an alcohol (e.g., ethanol) that may also function as a CPE;

(3) Layer 3 is a semi-permeable microporous membrane composed of, e.g., a polymer (e.g., polyethylene);

(4) Layer 4 is a peripheral skin-adhesive area, which can be composed of, e.g., acrylic;

(5) Layer 5 is a disc that seals the central gel-based drug reservoir (layer 2), and is attached to and removed with the release liner (layer 6); and

(6) Layer 6 is a release liner that can be, e.g., a silicone-coated polyester film.

The RTP of FIG. 1 can be made with heat-sealable components. For example, the gel-based drug reservoir can be packaged by heat seal between the backing layer and the microporous membrane. The release liner that protects the drug reservoir and the peripheral skin-adhesive area can be peeled off immediately prior to application of the RTP.

In other embodiments, an RTP comprises:

(1) a backing layer composed of, e.g., pigmented polyester and aluminum film;

(2) a drug reservoir containing one or more drugs and, e.g., mineral oil, polyisobutylene and colloidal silicon dioxide;

(3) a rate-controlling, microporous membrane composed of, e.g., polypropylene;

(4) a skin-adhesive layer that optionally contains one or more drugs and, e.g., mineral oil, polyisobutylene and colloidal silicon dioxide for quicker drug saturation of the skin; and

(5) a protective slit-release liner composed of, e.g., polyester which covers the skin-adhesive layer and is removed immediately prior to use.

In some embodiments, an RTP comprises a solution of one or more drugs in a volatile solvent, and optionally a non-volatile solvent. The liquid-based drug reservoir can optionally contain one or more CPEs, such as volatile CPEs (e.g., essential/volatile oils such as caraway oil, cardamom oil and lemon oil). Evaporative delivery of the drug(s) can form a drug depot in the skin (e.g., in the stratum corneum) which releases the drug(s) over a period of, e.g., about 2, 3 or 4 days or longer. To facilitate evaporative delivery of the drug(s), the skin-contacting adhesive layer can be peripheral and not cover the semi-permeable membrane if used. If the RTP has a skin-contacting adhesive layer rather than a semi-permeable membrane covering the liquid-based drug reservoir, the drug reservoir can contain a non-volatile or viscous solvent to prevent leakage of the content of the drug reservoir.

U.S. Pat. No. 9,289,397 describes reservoir-type transdermal patches that are divisible into one or more patch units and allow a subject to adjust the amount of drug(s) delivered depending on the number of patch units applied to the skin, as described in greater detail below.

3. Matrix-Type Transdermal Patches

In other embodiments, treprostinil or a salt thereof, and optionally an additional therapeutic agent, are delivered from a matrix-type transdermal patch (MTP). In some embodiments, an MTP comprises an impermeable backing layer/film, a matrix containing one or more drugs, and optionally a skin-contacting adhesive layer distinct from (e.g., surrounding or covering) the drug matrix. Alternatively, the drug matrix can be designed to adhere to the skin. To prevent detachment of the patch from the skin, a separate, protective adhesive layer (e.g., a medical/surgical tape or a PatchProtect™ or Tegaderm™ dressing) can optionally be applied over the entire patch and beyond the edges of the patch. MTPs generally have a protective release liner that covers the skin-contacting side of the patch and is removed immediately prior to use. In some embodiments, the backing layer is occlusive, which promotes hydration of the outer layer of the skin. The drug matrix can optionally contain one or more chemical permeation enhancers. In some embodiments, the matrix is a polymer matrix in which one or more drugs are substantially homogeneously dispersed or impregnated, and from which the drug(s) can be continuously released into the skin. The polymer matrix can be designed to control diffusion of the drug(s). The rate of drug delivery from a polymer matrix of a drug-in-adhesive patch or other type of MTP can be substantially constant (substantially zero-order kinetics) and can be controlled by, e.g., the formulation and the thickness of the drug/polymer matrix, such as the polymer(s) used and the amount thereof, the amount and the solubility of the drug(s), and any other excipient(s) used and the amount thereof. The amount of drug delivered from an MTP can depend on, e.g., the amount of drug in the matrix and the area of the patch applied to the skin. Advantages of an MTP, particularly a drug-in-adhesive patch, over an RTP can include, e.g., reduced thickness and weight, greater flexibility, better conformity and adhesion to the skin, greater patient comfort, and greater simplicity and lower cost in manufacturing.

The polymer matrix can be solid or semisolid, and can be composed of hydrophilic or/and hydrophobic/lipophilic polymer(s). The polymers can be linear or cross-linked, and can be adhesive or non-adhesive. Examples of polymers of which the polymer matrix can be composed include without limitation natural celluloses and cellulose derivatives (e.g., methyl cellulose, ethyl cellulose and cellulose esters), gums, gelatin, zein, polyacrylic acid, polyacrylate, polymethacrylic acid, polymethacrylate, polymethacrylate-co-siloxane, polyacrylamide, polyethylene glycol, polyvinyl alcohol, ethylene-vinyl alcohol, ethylene-vinyloxyethanol, polyvinylpyrrolidone, polyamides, polyureas, natural and synthetic rubbers (e.g., hydrin rubber, neoprene, nitrile rubber and silicon rubber), shellac, waxes, polybutadiene, polyethylene, polyisobutylene, polypropylene, chlorinated polyethylene, polyvinylchloride, polyvinylidene chloride, polyethylene-co-propylene, polyethylene-co-ethylacrylate, ethylene-vinyl acetate, vinyl chloride-vinyl acetate, polyacrylic esters (e.g., polymethylacrylate and polyethylacrylate), polymethylmethacrylate, nitrile-containing polymers, silicone-based polymers (e.g., polydimethylsiloxane and polyvinylsiloxane), and copolymers and combinations thereof. In certain embodiments, the polymer matrix is composed of a linear or cross-linked acrylic polymer (e.g., polyacrylic acid, polyacrylate or polymethacrylate), polyisobutylene, ethylene-vinyl acetate or a silicone polymer (e.g., polydimethylsiloxane or polyvinylsiloxane), or any combination thereof. To promote crosslinking of polymer(s) if desired, the polymer matrix can optionally contain a cross-linking agent (e.g., tetrapropoxy silane). Furthermore, the polymer matrix can optionally contain a plasticizer. As a non-limiting example, a semisolid suspension of drug cells can be formed in a polymer matrix comprising an acrylic polymer, a silicone adhesive and one or more drugs.

In some embodiments, an MTP comprises a water-swellable polymer matrix that can provide controlled- or/and sustained-release of one or more substantially water-soluble drugs from the patch. The water-swellable polymer matrix can be, e.g., a hydrogel. In certain embodiments, the polymer matrix comprises or is composed of one or more polysaccharides {e.g., a natural or synthetic gum (e.g., guar gum or tragacanth) or a natural or semi-synthetic cellulose (e.g., hypromellose [hydroxypropyl methylcellulose or HPMC] or carboxymethyl cellulose [CMC])} or/and one or more hydrophilic synthetic polymers (e.g., polyacrylic acid, polyacrylate, polymethacrylate, polyacrylamide, polyethylene glycol, or polyvinylpyrrolidone) that can be linear or cross-linked. Absorption of water (e.g., from the skin) by the drug-loaded polymer matrix results in swelling of the polymer matrix, which promotes diffusion of the drug(s) out of the matrix. The water absorption and swelling mechanism controls release of the drug(s) from the polymer matrix. To facilitate absorption of water from the skin by the drug-loaded polymer matrix, the polymer matrix can be a skin-contacting adhesive layer in a single-layer drug-in-adhesive patch (described below), or the patch can have a skin-contacting adhesive layer that surrounds and does not cover the polymer matrix.

U.S. Pat. No. 9,289,397 discloses a list of drug-permeable adhesives. In some embodiments, an adhesive layer, or each adhesive layer, through which one or more drugs diffuse, whether the adhesive layer is loaded with the drug(s) or not in the manufacturing of the patch, is a pressure-sensitive adhesive (PSA). In further embodiments, each adhesive layer of a patch is a PSA. Examples of polymers of which PSAs can be composed include without limitation polyisobutylene, polyvinylacetate, acrylic polymers (e.g., cyanoacrylates, polyacrylate, polymethacrylate and esters thereof [e.g., polymethylacrylate and polybutylacrylate]), hydrogels (e.g., polyethylene glycol, polyvinylpyrrolidone, polyglutamic acid and gelatin), silicone polymers (e.g., polydimethylsiloxane, silicone-2675 and silicone-2920), natural and synthetic rubbers (e.g., butyl rubber), polysaccharides (e.g., chitosan, pectin, natural and synthetic gums [e.g., algin, guar gum, karaya gum and tragacanth], and natural and semi-synthetic celluloses [e.g., starch, CMC and HPMC]), polypeptides (e.g., fibrin), and combinations thereof. In certain embodiments, a PSA is composed of polyisobutylene, an acrylic polymer (e.g., polyacrylate) or a silicone polymer (e.g., silicone-2675 or silicone-2920), or any combination thereof. An adhesive layer can optionally contain a tackifier (e.g., a rosin ester).

In some embodiments, an MTP comprises a backing layer, a matrix (e.g., a polymer matrix) containing one or more drugs, and a peripheral skin-contacting adhesive layer that does not overlap or cover the drug matrix. In other embodiments, an MTP comprises a backing layer, a matrix (e.g., a polymer matrix) containing one or more drugs, and a skin-contacting adhesive layer that overlaps or covers the drug matrix.

In certain embodiments, an MTP has the structure depicted in FIG. 2. The MTP of FIG. 2 has the following layers:

(1) Layer 1 is a protective release liner covering the patch that is peeled off immediately prior to application of the patch to the skin;

(2) Layer 2 is an adhesive polymer matrix containing one or more drugs;

(3) Layer 3 is a layer separating the adhesive film of Layer 4 from the polymer matrix;

(4) Layer 4 is an overlay adhesive film; and

(5) Layer 5 is a backing layer.

In further embodiments, an MTP is a single-layer drug-in-adhesive (DIA) patch comprising an impermeable backing layer and a skin-contacting adhesive layer incorporating one or more drugs in a polymeric or viscous adhesive. The single-layer DIA patch can optionally have a peripheral, non-drug-loaded, skin-contacting adhesive layer. In some embodiments, the adhesive of the drug-loaded, skin-contacting adhesive layer (and the optional peripheral, non-drug-loaded, skin-contacting adhesive layer) is a pressure-sensitive adhesive (PSA). In certain embodiments, the PSA is composed of an acrylic polymer (e.g., polyacrylate), a polyalkylene (e.g., polyisobutylene) or a silicone-based polymer (e.g., silicone-2675 or silicone-2920), or any combination thereof.

The amount of drug delivered from a single-layer DIA patch is controlled by drug diffusion through the adhesive layer and is directly proportional to the surface area of the patch in contact with the skin. In addition to adhering to the skin, the adhesive layer of a DIA patch can be designed to control the delivery rate of the drug(s). If a drug is completely dissolved in the adhesive, the rate of drug release from the DIA patch depends on the drug concentration in the adhesive (first-order kinetics), and the drug release rate may decline with wear time unless a high percentage (e.g., about 80%) of the initial drug loading remains in the patch when the patch is removed or replaced. On the other hand, the release rate of a drug suspended in the adhesive can be substantially constant (substantially zero-order kinetics) throughout the wear time because as the drug is released from the DIA patch and absorbed into the skin, more suspended drug dissolves in the adhesive. The polymer matrix of the adhesive can also be designed to provide a barrier to drug release, such as the polymer(s) used and the amount or ratio thereof, the use of cross-linked polymer(s), and the viscosity of the polymer(s).

If a drug is insoluble in an adhesive (e.g., a silicone adhesive), sustained delivery of the drug from a DIA patch can be achieved through the use of an amphiphilic solvent. Dissolution of the drug in an amphiphilic solvent (e.g., an alcohol, such as ethanol or/and 1-octanol or 2-octanol) and even dispersion of the drug-loaded solvent in the adhesive matrix may form an emulsion in the matrix and can provide sustained delivery of the drug from the DIA patch for up to about 72 hours or longer. A DIA patch can also be designed so that body heat (about 37° C.) induces liquefaction of the material in the adhesive, dissolution of the drug(s) embedded in the adhesive, and diffusion of the drug(s) into the skin.

A variation of a single-layer DIA patch has a rate-controlling adhesive layer. In some embodiments, a single-layer DIA patch with a rate-controlling adhesive layer comprises an impermeable backing layer, an adhesive layer incorporating one or more drugs in a polymeric or viscous adhesive (e.g., a PSA such as polyacrylate or polyisobutylene), and a skin-contacting, rate-controlling adhesive layer overlapping or covering the drug-loaded adhesive layer. The drug-loaded adhesive layer and the rate-controlling adhesive layer can contain the same or different polymeric or viscous adhesive. The permeability and thickness of the non-drug-loaded, skin-contacting, rate-controlling adhesive layer can be selected so that it controls diffusion/release of the drug(s) from the patch.

In additional embodiments, an MTP is a multi-layer DIA patch containing a plurality of drug-loaded adhesive layers. In some embodiments, the multi-layer DIA patch comprises an impermeable backing layer and 2, 3, 4, 5 or more adhesive layers each loaded with one or more drugs, wherein the loading of the drug(s) varies progressively (e.g., incrementally) from DIA layer to DIA layer. Because such a patch contains a gradient of drug loading along the diffusional path across the multilaminate, drug-loaded adhesive layers, the patch can deliver the drug(s) at a substantially constant rate (substantially zero-order kinetics). Such a patch can be called a multi-layer, drug-gradient DIA patch. In certain embodiments, the closer to the skin an adhesive layer is, the lower the drug loading in that adhesive layer. In other embodiments, the closer to the skin an adhesive layer is, the higher the drug loading in that adhesive layer. A multi-layer DIA patch can also have a substantially similar drug loading in each of the adhesive layers. The thickness of the drug-loaded adhesive layers can be substantially similar or can vary (e.g., progressively or incrementally). In certain embodiments, the thickness of each of the drug-loaded adhesive layers is substantially similar. The adhesive layers can contain the same or different polymeric or viscous adhesive. In certain embodiments, the adhesive layers each contain the same adhesive. In a variation of a multi-layer, drug-gradient DIA patch, the patch has a non-drug-loaded, skin-contacting, rate-controlling adhesive layer, similar to that described above for a single-layer DIA patch with a rate-controlling adhesive layer.

Anther type of multi-layer DIA patch contains a rate-controlling semi-permeable membrane. In some embodiments, the multi-layer DIA patch comprises an impermeable backing layer, an upper adhesive layer containing one or more drugs, a semi-permeable or microporous membrane, and a skin-contacting adhesive layer containing the drug(s). The multi-layer DIA patch can optionally have a peripheral, non-drug-loaded, skin-contacting adhesive layer. The skin-contacting adhesive layer is designed for immediate or initial release of the drug(s) while the second (upper) adhesive layer is designed for controlled release of the drug(s) through the semi-permeable membrane. U.S. Pat. No. 9,289,397 discloses a list of polymers of which semi-permeable membranes can be composed. The upper adhesive layer and the skin-contacting adhesive layer can contain the same or different polymeric or viscous adhesive. In certain embodiments, the upper adhesive layer and the skin-contacting adhesive layer contain the same adhesive. A variation of this type of multi-layer DIA patch has another type of release-limiting (e.g., rate-controlling) layer instead of a semi-permeable membrane between the two drug-loaded adhesive layers, such as a polymeric layer or an adhesive layer. Another variation of this type of multi-layer DIA patch has no semi-permeable membrane or other release-limiting layer between the two drug-loaded adhesive layers.

Another type of MTP is a hybrid of an RTP and an MTP. In some embodiments, a membrane/matrix hybrid MTP comprises an impermeable backing layer, a polymer matrix containing one or more drugs, a semi-permeable or microporous membrane that controls drug release, and a skin-contacting adhesive layer that can surround or cover the semi-permeable membrane. The polymer matrix (e.g., polyisobutylene) may or may not be an adhesive, and can be designed to control diffusion of the drug(s) too.

An additional type of MTP is a microreservoir MTP. In some embodiments, a microreservoir MTP comprises an impermeable backing layer, a polymer matrix containing a plurality of microscopic drug reservoirs (drug microreservoirs), and a skin-contacting adhesive layer that can surround or cover the drug/polymer matrix. The microreservoir MTP can optionally have a semi-permeable or microporous membrane that controls drug release from the polymer matrix. In some embodiments, drug microreservoirs are formed by suspending one or more drugs in an aqueous solution containing a water-miscible drug solubilizer (e.g., polyethylene glycol). The drug suspension is homogenously dispersed (e.g., by high-shear mechanical force) in a lipophillic polymer to form thousands of microscopic drug reservoirs. The dispersion is quickly stabilized by immediately crosslinking the polymer chains in situ to form a medicated polymer disc of a certain area and thickness, which can be mounted onto an adhesive pad. In other embodiments, drug microreservoirs are formed by suspending one or more drugs in an aqueous solution containing a water-miscible drug solubilizer (e.g., polyethylene glycol) and dispersing the drug(s) with a dispersing agent (e.g., isopropyl palmitate) in a viscous polymer (e.g., a silicone elastomer). The drug/polymer dispersion is quickly molded to form a medicated polymer disc containing thousands of microscopic drug reservoirs in situ on an impermeable backing layer (e.g., a metallic plastic laminate) by injection molding under instantaneous heating. The rate of drug release from a microreservoir MTP can be controlled by dissolution and diffusion of the drug(s) in and through the polymer matrix, and through the semi-permeable membrane if used, and can be substantially constant (substantially zero-order kinetics).

A further type of MTP contains polymer-coated drug microparticles. In some embodiments, such an MTP comprises an impermeable backing layer, a polymer matrix containing a plurality of polymer-coated drug microparticles, and optionally a skin-contacting adhesive layer that can surround or cover the drug/polymer matrix. Alternatively, the drug/polymer matrix can be a skin-contacting adhesive layer. In some embodiments, one or more drugs are coated with a hydrophilic polymer (e.g., polyethylene glycol, polymethacrylate or a natural or semi-synthetic cellulose) to form polymer-coated drug microparticles with a coating thickness of, e.g., about 1-200 microns (e.g., about 1-100 microns or 100-200 microns). The polymer-coated drug microparticles are dispersed in a polymer matrix comprising or composed of, e.g., a polymeric gellant (e.g., gelatin) or/and a water-absorbing polymer (e.g., a water-swellable polymer described above). Absorption of water (e.g., from the skin) by the polymer matrix promotes dissolution of the polymeric coating of the drug microparticles, whose rate can depend on, e.g., the aqueous solubility and the thickness of the polymeric coating. Absorption of water by the polymer matrix and dissolution of the polymeric coating provide controlled- and sustained-release of the drug(s) from the patch. To facilitate absorption of water from the skin by the polymer matrix, the polymer matrix can be a skin-contacting adhesive layer in a single-layer DIA patch, or the patch can have a skin-contacting adhesive layer that surrounds and does not cover the polymer matrix.

The amount of drug delivered from an MTP can depend on, e.g., the amount of drug loaded in the patch and the area of the patch applied to the skin. Therefore, different doses of a drug can be delivered from a plurality of patches of the same type of MTP which have, e.g., different skin-contacting surface areas over which the drug is delivered or/and different amounts of the drug loaded in the patches. Alternatively, an MTP can be cut (e.g., with a scissors) into a smaller piece so that the cut patch delivers a smaller amount of the drug. An MTP can be manufactured with demarcations so that the patch can be easily and accurately torn by hand or cut with a scissors into two or more smaller pieces that deliver a lower dose or a particular dose of the drug. Similarly, a strip or roll of an MTP can be manufactured with demarcated areas for different drug doses that can be easily torn by hand or cut with a scissors so that a patient can select the size of the patch to apply to the skin for delivery of a particular dose of the drug. Types of MTP that can be cut into a smaller piece for delivery of a lower dose or a selected dose of a drug include without limitation a single-layer DIA patch without or with a non-drug-loaded, skin-contacting, rate-controlling adhesive layer and a patch comprising a drug-loaded polymer matrix and a skin-contacting adhesive layer covering the drug-loaded polymer matrix.

U.S. Pat. No. 9,289,397 describes transdermal patches that allow for titration/adjustment of the drug dosage. A parent/mother patch comprises a plurality of patch units that are connected to each other along one or more borders of the patch units. Each patch unit is surrounded by borders (e.g., on all sides or along all edges), is defined by one or more lines of separation (e.g., lines of perforation or lines of weakness of the backing layer) along one or more borders, and comprises an impermeable backing layer and a drug layer. The borders of each patch unit can optionally have a skin-contacting adhesive layer. The lines of separation can be, e.g., parallel or/and perpendicular to each other, and can be spaced at regular intervals. The parent patch, or the plurality of patch units, is divisible into 1, 2, 3, 4, 5 or more separate patch units, such as by tearing by hand or cutting with a scissors along the one or more lines of separation. The amount of drug(s) delivered to a subject is proportional to the number of patch units applied to the skin, and thus is titratable/adjustable by the subject under the direction of the treating physician. For example, if each patch unit is designed to deliver about 0.5 mg of a drug per day over a certain time period, then application of 1, 2, 3, 4 or 5 patch units to the skin would deliver about 0.5, 1, 1.5, 2 or 2.5 mg, respectively, of the drug per day over that time period. The parent patch can, e.g., comprise 1, 2 or more rows of patch units, or be in a 2×2 format (2 rows and 2 columns of patch units). FIGS. 1, 2 and 3a-3d of U.S. Pat. No. 9,289,397 show embodiments of dosage-titratable transdermal patches. The dosage-titratable transdermal patches can be matrix-type transdermal patches or reservoir-type transdermal patches.

4. Other Matrix-Containing Transdermal Release Systems

In additional embodiments, treprostinil or a salt thereof, and optionally an additional therapeutic agent, are contained or dispersed in a matrix material. The matrix material can comprise a polymer (e.g., ethylene-vinyl acetate) and can control the release of the therapeutic agent(s) by controlling dissolution or/and diffusion of the therapeutic agent(s) in or from, e.g., a reservoir (the matrix material can be designed to serve as the drug reservoir), and can enhance the stability of the therapeutic agent(s) while contained in the reservoir. In certain embodiments, the matrix is contained in or functions as the drug reservoir. The matrix-containing “release system” can be configured as a transdermal patch and can contain an excipient that can accelerate the therapeutic agent(s)′ release, such as a water-swellable material (e.g., a hydrogel) that aids in expelling the therapeutic agent(s) out of the reservoir. U.S. Pat. Nos. 4,144,317 and 5,797,898 describe examples of such a release system.

The release system can provide a temporally modulated release profile (e.g., pulsatile release) when time variation in plasma levels is desired, or a more continuous or consistent release profile when a constant plasma level is desired. Pulsatile release can be achieved from an individual reservoir or from a plurality of reservoirs. For example, where each reservoir provides a single pulse, multiple pulses (“pulsatile” release) are achieved by temporally staggering the single-pulse release from each of multiple reservoirs. Alternatively, multiple pulses can be achieved from a single reservoir by incorporating several layers of a release system and other materials into a single reservoir. Continuous release can be achieved by incorporating a release system that degrades, dissolves, or allows diffusion of a therapeutic agent through it over an extended time period. In addition, continuous release can be approximated by releasing several pulses of a therapeutic agent in rapid succession (“digital” release). An active release system can be used alone or in conjunction with a passive release system, as described in U.S. Pat. No. 5,797,898.

### VII. ADDITIONAL THERAPEUTIC AGENTS

In addition to treprostinil or a salt thereof, a composition (e.g., a topical composition), device (e.g., a patch) or system (collectively called a transdermal delivery system [TDS] here for simplicity) can optionally comprise and deliver one or more additional therapeutic agents. Alternatively, one or more additional therapeutic agents can optionally be administered separately from (concurrently with, before or after administration of) treprostinil or a salt thereof.

In some embodiments, the one or more additional therapeutic agents have a local effect and are used, e.g., to prevent or reduce any side effect associated with dermal or transdermal administration of treprostinil or a salt thereof, such as pain, skin irritation (e.g., erythema) or edema. In other embodiments, the one or more additional therapeutic agents have a systemic effect and are used, e.g., to treat a treprostinil-responsive medical condition, such as pulmonary hypertension (including PAH).

In some embodiments, a TDS comprises a local anesthetic. The local anesthetic can be used, e.g., to prevent any pain that may result from the application of a physical enhancement technique. Non-limiting examples of local anesthetics include amides (e.g., articaine, bupivacaine, cinchocaine [dibucaine], etidocaine, levobupivacaine, lidocaine, mepivacaine, prilocaine, ropivacaine and trimecaine), esters (e.g., benzocaine, chloroprocaine, cocaine, cyclomethycaine, dimethocaine [larocaine], piperocaine, procaine [novocaine], proparacaine, propoxycaine, stovaine and tetracaine [amethocaine]), ethers (e.g., polidocanol and pramocaine [pramoxine]), naturally derived local anesthetics (e.g., cocaine, eugenol, menthol, saxitoxin, neosaxitoxin and tetrodotoxin), and analogs, derivatives and salts thereof. In certain embodiments, the local anesthetic is selected from articaine, benzocaine, bupivacaine, dimethocaine, lidocaine, mepivacaine, prilocaine, procaine, ropivacaine, tetracaine, trimecaine and combinations thereof.

Alternative to or in addition to a local anesthetic, a TDS can comprise an analgesic. In certain embodiments, the analgesic has a local effect. Non-limiting examples of analgesics include:

acetaminophen (paracetamol);

non-steroidal anti-inflammatory drugs (NSAIDs), such as acetic acid derivatives (e.g., diclofenac), propionic acid derivatives (e.g., ibuprofen and naproxen), salicylates (e.g., aspirin), and selective COX-2 inhibitors (e.g., celecoxib and etoricoxib);

alcohols, such as ethanol;

opioids, such as morphine, dihydromorphine, codeine, hydrocodone, oxycodone, pethidine, buprenorphine, tapentadol and tramadol;

analgesics that may have psychotropic property, such as NMDA receptor antagonists (e.g., dextromethorphan and ketamine) and alpha- (e.g., α₂-) adrenoreceptor agonists (e.g., clonidine);

antidepressants, such as tricyclic antidepressants (e.g., amitriptyline, amitriptylinoxide, amoxapine, clomipramine, dosulepin [dothiepin], doxepin and melitracen) and serotonin-norepinephrine reuptake inhibitors (e.g., bicifadine, duloxetine, milnacipran, levomilnacipran, sibutramine, tramadol, tapentadol, venlafaxine, desvenlafaxine and SEP-227162);

other atypical analgesics, such as anticonvulsants (e.g., carbamazepine, gabapentin, pregabalin, and valproic acid and salts thereof [e.g., sodium valproate]), voltage-gated sodium channel blockers (e.g., funapide, mexiletine, nefopam, orphenadrine and raxatrigine), and neuronal potassium channel openers (e.g., flupirtine); and analogs, derivatives and salts thereof.

In some embodiments, a TDS comprises a local anesthetic or/and an analgesic, and one or more chemical permeation enhancers (CPEs). In certain embodiments, the one or more CPEs are selected from N-lauroyl sarcosine, sodium lauroyl sarcosinate, sodium lauryl sulfoacetate, sodium octyl sulfate, methyl laurate, isopropyl myristate, oleic acid and glyceryl oleate.

In further embodiments, a TDS comprises an anti-inflammatory agent, which may or may not also have an analgesic effect. In certain embodiments, the anti-inflammatory agent has a local effect. The anti-inflammatory agent can be used, e.g., to reduce any skin irritation (e.g., erythema, which may be a result of an inflammatory process) or/and any pain associated with dermal or transdermal administration of treprostinil or a salt thereof. An anti-inflammatory agent (e.g., a corticosteroid/glucocorticoid or an H₁ antihistamine) can also be used to reduce any allergic skin reaction or skin irritation that may be caused by an adhesive of a transdermal patch.

In some embodiments, the anti-inflammatory agent is a non-steroidal anti-inflammatory drug (NSAID). Examples of NSAIDs include without limitation:

acetic acid derivatives, such as aceclofenac, bromfenac, diclofenac, etodolac, indomethacin, ketorolac, nabumetone, sulindac, sulindac sulfide, sulindac sulfone and tolmetin;

anthranilic acid derivatives (fenamates), such as flufenamic acid, meclofenamic acid, mefenamic acid and tolfenamic acid;

enolic acid derivatives (oxicams), such as droxicam, isoxicam, lornoxicam, meloxicam, piroxicam and tenoxicam;

propionic acid derivatives, such as fenoprofen, flurbiprofen, ibuprofen, dexibuprofen, ketoprofen, dexketoprofen, loxoprofen, naproxen and oxaprozin;

salicylates, such as diflunisal, salicylic acid, acetylsalicylic acid (aspirin), choline magnesium trisalicylate, and salsalate;

COX-2-selective inhibitors, such as apricoxib, celecoxib, etoricoxib, firocoxib, fluorocoxibs (e.g., fluorocoxibs A-C), lumiracoxib, mavacoxib, parecoxib, rofecoxib, tilmacoxib (JTE-522), valdecoxib, 4-O-methylhonokiol, niflumic acid, DuP-697, CG100649, GW406381, NS-398, SC-58125, benzothieno[3,2-d]pyrimidin-4-one sulfonamide thio-derivatives, and COX-2 inhibitors derived from Tribulus terrestris;

other kinds of NSAIDs, such as monoterpenoids (e.g., eucalyptol and phenols [e.g., carvacrol]), anilinopyridinecarboxylic acids (e.g., clonixin), sulfonanilides (e.g., nimesulide), and dual inhibitors of lipooxygenase (e.g., 5-LOX) and cyclooxygenase (e.g., COX-2) (e.g., chebulagic acid, licofelone, 2-(3,4,5-trimethoxyphenyl)-4-(N-methylindol-3-yl)thiophene, and di-tert-butylphenol-based compounds [e.g., DTPBHZ, DTPINH, DTPNHZ and DTPSAL]); and

analogs, derivatives and salts thereof.

In other embodiments, the anti-inflammatory agent is a corticosteroid (e.g., a glucocorticoid). Non-limiting examples of corticosteroids include hydrocortisone types (e.g., cortisone and derivatives thereof [e.g., cortisone acetate], hydrocortisone and derivatives thereof [e.g., hydrocortisone acetate, hydrocortisone-17-aceponate, hydrocortisone-17-buteprate, hydrocortisone-17-butyrate and hydrocortisone-17-valerate], prednisolone, methylprednisolone and derivatives thereof [e.g., methylprednisolone aceponate], prednisone, and tixocortol and derivatives thereof [e.g., tixocortol pivalate]), betamethasone types (e.g., betamethasone and derivatives thereof [e.g., betamethasone benzoate, betamethasone dipropionate, betamethasone sodium phosphate and betamethasone valerate], dexamethasone and derivatives thereof [e.g., dexamethasone sodium phosphate], and fluocortolone and derivatives thereof [e.g., fluocortolone caproate and fluocortolone pivalate]), halogenated steroids (e.g., alclometasone and derivatives thereof [e.g., alclometasone dipropionate], beclometasone and derivatives thereof [e.g., beclometasone dipropionate], clobetasol and derivatives thereof [e.g., clobetasol-17-propionate], clobetasone and derivatives thereof [e.g., clobetasone-17-butyrate], desoximetasone and derivatives thereof [e.g., desoximetasone acetate], diflorasone and derivatives thereof [e.g., diflorasone diacetate], diflucortolone and derivatives thereof [e.g., diflucortolone valerate], fluprednidene and derivatives thereof [e.g., fluprednidene acetate], fluticasone and derivatives thereof [e.g., fluticasone propionate], halobetasol [ulobetasol] and derivatives thereof [e.g., halobetasol proprionate], halometasone and derivatives thereof [e.g., halometasone acetate], mometasone and derivatives thereof [e.g., mometasone furoate], and triamcinolone and derivatives thereof [e.g., triamcinolone diacetate]), acetonides and related substances (e.g., amcinonide, budesonide, ciclesonide, desonide, flunisolide, fluocinonide, fluocinolone acetonide, flurandrenolide [flurandrenolone or fludroxycortide], halcinonide and triamcinolone acetonide), carbonates (e.g., prednicarbate), and analogs, derivatives and salts thereof.

In certain embodiments, the corticosteroid is selected from betamethasone, clobetasol, halobetasol, halobetasol propionate, triamcinolone acetonide, and combinations thereof.

In some embodiments, the anti-inflammatory agent is a corticosteroid/glucocorticoid of moderate or medium potency, which can be formulated for topical use. Examples of corticosteroids having moderate or medium potency include Groups III, IV and V corticosteroids under the 7-group US classification system and Class II corticosteroids under the 4-class European classification system, where the potency of a corticosteroid can depend on, e.g., its concentration and the type of topical composition:

Group III US (upper mid-strength), including but not limited to amcinonide 0.05-0.1%, betamethasone dipropionate 0.05%, betamethasone valerate 0.1%, diflorasone diacetate 0.05%, fluocinonide 0.05%, fluticasone propionate 0.005%, halometasone 0.05%, mometasone furoate 0.1%, triamcinolone acetonide 0.5%, and triamcinolone diacetate 0.5%;

Group IV US (mid-strength), including but not limited to desoximetasone 0.05%, fluocinolone acetonide 0.025-0.2%, clocortolone pivalate 0.1%, flurandrenolide 0.05%, hydrocortisone butyrate 0.1%, hydrocortisone probutate 0.1%, hydrocortisone valerate 0.2%, mometasone furoate 0.1%, and triamcinolone acetonide 0.1-0.5%;

Group V US (lower mid-strength), including but not limited to betamethasone dipropionate 0.05%, betamethasone valerate 0.1%, desonide 0.05%, fluocinolone acetonide 0.025/0.03%, fluocinolone acetonide 0.01%, flurandrenolide 0.025-0.05%, fluticasone propionate 0.05%, hydrocortisone butyrate 0.1%, hydrocortisone probutate 0.1%, hydrocortisone valerate 0.2%, prednicarbate 0.1%, and triamcinolone acetonide 0.1%; and

Class II EU (moderate), including but not limited to clobetasone butyrate 0.05% and triamcinolone acetonide 0.1-0.5%.

In further embodiments, a TDS comprises an antihistamine. In certain embodiments, the antihistamine has a local effect. Histamine, which is secreted by, e.g., mast cells prevalent in the skin, promotes inflammation and increases vascular permeability, which causes fluid to leak from capillaries into tissues. Antihistamines suppress histamine-induced inflammation, vasodilation and edema. In some embodiments, the antihistamine inhibits action at the histamine H₁ receptor. Examples of H₁ antihistamines include without limitaiton acrivastine, antazoline, astemizole, azatadine, azelastine, bepotastine, bilastine, bromodiphenhydramine, brompheniramine, buclizine, carbinoxamine, cetirizine, chlorcyclizine, chlorodiphenhydramine, chlorpheniramine (chlorphenamine), chlorpromazine, chloropyramine, cidoxepin, clemastine, cyclizine, cyproheptadine, desloratadine, dexbrompheniramine, dexchlorpheniramine, dimenhydrinate, dimetindene, diphenhydramine, doxepin, doxylamine, ebastine, embramine, esmirtazapine [(S)-(+)-mirtazapine], fexofenadine, hydroxyzine, ketotifen, levocabastine, levocetirizine, loratadine, meclozine (meclizine), mepyramine, mirtazapine, mizolastine, olopatadine, orphenadrine, phenindamine, pheniramine, phenyltoloxamine, promethazine, pyrilamine, quetiapine, quifenadine, rupatadine, terfenadine, trimeprazine [alimemazine], tripelennamine, triprolidine, and analogs, derivatives and salts thereof. In certain embodiments, the antihistamine is a second-generation or third-generation H₁ antihistamine, which is much more selective for peripheral H₁ receptors than for central nervous system H₁ receptors and cholinergic receptors. Non-limiting examples of second-generation and third-generation H₁ antihistamines include acrivastine, astemizole, azelastine, bepotastine, bilastine, cetirizine, cidoxepin, levocetirizine, ebastine, fexofenadine, levocabastine, loratadine, desloratadine, mizolastine, olopatadine, quifenadine, rupatadine, terfenadine, and analogs, derivatives and salts thereof.

In some embodiments, a TDS comprises an anti-inflammatory agent (e.g., an NSAID, a corticosteroid or an H₁ antihistamine, or any combination thereof) and one or more CPEs. In certain embodiments, the one or more CPEs are selected from N-lauroyl sarcosine, sodium lauroyl sarcosinate, sodium lauryl sulfoacetate, sodium octyl sulfate, methyl laurate, isopropyl myristate, oleic acid and glyceryl oleate.

In additional embodiments, a TDS comprises a vasoconstrictor that has a local effect. Local vasoconstriction can reduce erythema, which can be caused by dilation of capillaries. Furthermore, co-administration of a local vasoconstrictor with, e.g., a local anesthetic can increase the duration of local anesthesia by constricting blood vessels, thereby safely concentrating the anesthetic agent for an extended duration. Examples of vasoconstrictors include without limitation:

antihistamines, such as H₁ antihistamines;

stimulants, such as substituted phenethylamines (e.g., amphetamine, dextroamphetamine, lisdexamfetamine, methamphetamine, dextromethamphetamine, levomethamphetamine, mephedrone, methylphenidate, dexmethylphenidate, ephedrine, pseudoephedrine, phenylpropanolamine and prolintane), methamphetamine analogs (e.g., propylhexedrine), ampakines (e.g., ampalex and CX7171), eugeroics (e.g., adrafinil, armodafinil, hydrafinil and modafinil), caffeine, cocaine, nicotine and methylhexanamine (dimethylamylamine);

decongestants, such as alpha (e.g., α₁ or/and α₂) adrenergic receptor agonists (e.g., naphazoline, oxymetazoline, phenylephrine, synephrine, tetryzoline [tetrahydrozoline], tramazoline and xylometazoline); and

analogs, derivatives and salts thereof.

In some embodiments, a TDS comprises a local vasoconstrictor (e.g., an H₁ antihistamine, a stimulant or a decongestant, or any combination thereof) and one or more CPEs. In certain embodiments, the one or more CPEs are selected from N-lauroyl sarcosine, sodium lauroyl sarcosinate, sodium lauryl sulfoacetate, sodium octyl sulfate, methyl laurate, isopropyl myristate, oleic acid and glyceryl oleate.

In addition to treprostinil or a salt thereof, a TDS can comprise and deliver any combination of additional therapeutic agents (e.g., any combination of local anesthetic, analgesic, anti-inflammatory agent and local vasoconstrictor), optionally in conjunction with one or more chemical permeation enhancers.

In some embodiments, alternative to or in addition to delivery from a treprostinil-containing TDS (e.g., a transdermal patch), one or more additional therapeutic agents are administered separately from (concurrently with, before or after application of) the TDS. In some embodiments, one or more additional therapeutic agents are administered locally at or near the site of application of the treprostinil-containing TDS to prevent or reduce any local side effect that may be associated with application of the TDS, such as pain, skin irritation (e.g., erythema), allergic skin reaction or edema. Examples of such therapeutic agents include without limitation local anesthetics, analgesics, anti-inflammatory agents (including corticosteroids/glucocorticoids, NSAIDS and H₁ antihistamines), and local vasoconstrictors. In some embodiments, the one or more additional therapeutic agents are administered dermally or transdermally by applying a topical composition to the skin at or near the site of application of the treprostinil-containing TDS. Such a topical composition can be, e.g., a cream, a lotion, a gel, an ointment, a jelly, a paste, a liniment, a foam or a dermal spray. In some embodiments, a topical composition containing one or more additional therapeutic agents, or two or more topical compositions each containing one or more additional therapeutic agents, is/are applied to the skin, and then the treprostinil-containing TDS is applied at (e.g., on or over) or near the site of application of the one or more topical compositions. In certain embodiments, a topical composition (e.g., a cream) containing a corticosteroid/glucocorticoid of moderate or medium potency (e.g., 0.1% triamcinolone acetonide) is applied to the skin, and then the treprostinil-containing TDS is applied at (e.g., on or over) or near the site of application of the topical composition.

In further embodiments, one or more additional therapeutic agents administered separately from the treprostinil-containing TDS are administered systemically. In some embodiments, the one or more additional therapeutic agents are used to treat a treprostinil-responsive medical condition, such as pulmonary hypertension (including PAH). Examples of such therapeutic agents include without limitation vasoactive agents (including vasodilating agents), diuretics, anticoagulants and cardiac glycosides (described below). Certain therapeutic agents (e.g., analgesics and anti-inflammatory agents such as NSAIDS and H₁ antihistamines) that can be used to prevent or reduce any local side effect associated with application of the treprostinil-containing TDS can also be administered systemically. Routes of systemic administration include without limitation oral and parenteral (e.g., intravenous, intramuscular, subcutaneous, intranasal [e.g., by nasal spray or drop] and pulmonary [e.g., by oral or nasal inhalation]).

### VIII. THERAPEUTIC USES OF TREPROSTINIL

Treprostinil, a prostacyclin (prostaglandin I₂) analog, has a variety of prostacyclin-like effects. For example, treprostinil can promote vasodilation, inhibit platelet activation and aggregation, inhibit thrombus formation, stimulate thrombolysis, inhibit atherogenesis, inhibit cell proliferation, induce angiogenesis, promote endothelial cell membrane remodeling, reduce inflammation, and provide cytoprotection. Treprostinil and salts thereof can be dermally or transdermally administered to treat a wide variety of conditions, including without limitation: pulmonary hypertension, portopulmonary hypertension, pulmonary fibrosis, interstitial lung disease, ischemic diseases (e.g., myocardial ischemia, ischemic stroke, peripheral vascular disease [including peripheral arterial disease], ischemia of a limb, Raynaud's phenomenon [including Raynaud's disease and Raynaud's syndrome], scleroderma [including systemic sclerosis] and renal insufficiency), ischemic ulcers (e.g., digital ulcers), cardiovascular disease (e.g., coronary artery disease), heart failure (e.g., congestive heart failure), conditions requiring anticoagulation (e.g., post myocardial infarction and post cardiac surgery), atherogenesis (e.g., atherosclerosis), thrombotic microangiopathy, vein occlusion (e.g., central retinal vein occlusion), hypertension (e.g., preeclampsia), diabetic vasculopathy, extracorporeal circulation, inflammatory diseases (e.g., chronic obstructive pulmonary disease [COPD] and psoriasis), reproduction and parturition, conditions of unregulated cell growth (e.g., tumors and cancers), cell/tissue preservation, and other therapeutic areas where prostacyclin or treprostinil treatment may provide benefit.

In some embodiments, treprostinil or a pharmaceutically acceptable salt, solvate, hydrate, clathrate or polymorph thereof is dermally or transdermally administered to treat a prostacyclin- or treprostinil-responsive condition selected from pulmonary hypertension, pulmonary fibrosis, interstitial lung disease, asthma, congestive heart failure, peripheral vascular disease, severe intermittent claudication, atherogenesis (e.g., atherosclerosis), ischemic lesions (e.g., peripheral ischemic lesions on the skin, such as those caused by Buerger's disease, Raynaud's phenomenon, Raynaud's disease, scleroderma and systemic sclerosis), critical limb ischemia, ischemic ulcers (e.g., digital ulcers), skin ulcers, neuropathic foot ulcers (e.g., diabetic neuropathic foot ulcer), kidney malfunction and failure, immunosuppression, proliferative disorders (e.g., tumors and cancers, such as those of the head and neck, brain, lung, liver, kidney, pancreas, gastrointestinal tract [e.g., colon], prostate and breast), and pain associated with each of the preceding conditions.

Treprostinil or a salt thereof can be used in conjunction with an additional therapeutic agent to treat any condition responsive to treatment with prostacyclin or treprostinil. As a non-limiting example, to treat a vascular (e.g., cardiovascular) disorder treprostinil or a salt thereof can be used in combination with a vascular (e.g., cardiovascular) therapeutic, such as an antiplatelet agent, a phosphodiesterase inhibitor, a calcium channel blocker or an endothelial antagonist, or any combination thereof.

In some embodiments, treprostinil or a salt thereof is dermally or transdermally administered to treat pulmonary hypertension. An additional therapeutic agent (e.g., a vasoactive agent, a diuretic, an anticoagulant or a cardiac glycoside, or any combination thereof) can optionally be administered to treat pulmonary hypertension. In certain embodiments, the pulmonary hypertension is pulmonary arterial hypertension.

Pulmonary hypertension is an increase of blood pressure in the lung vasculature, including the pulmonary artery, pulmonary vein and pulmonary capillaries. Thus, pulmonary hypertension encompasses pulmonary arterial hypertension (PAH) and pulmonary venous hypertension (PVH) (e.g., congestive heart failure). More broadly, pulmonary hypertension encompasses:

WHO Group I—pulmonary arterial hypertension, including idiopathic PAH, heritable PAH (e.g., BMPR2, ALK1 and endoglin [with or without hereditary hemorrhagic telangiectasia]), drug- and toxin-induced PAH, PAH associated with various conditions (e.g., connective tissue disease, HIV infection, portal hypertension, congenital heart disease, schistosomiasis, and chronic hemolytic anemia [e.g., sickle cell disease]), persistent pulmonary hypertension of the newborn, pulmonary veno-occlusive disease (PVOD), and pulmonary capillary hemangiomatosis (PCH);

WHO Group II—pulmonary hypertension owing to left heart disease, including systolic dysfunction, diastolic dysfunction and valvular heart disease;

WHO Group III—pulmonary hypertension owing to lung disease or/and hypoxia, including chronic obstructive pulmonary disease (COPD), interstitial lung disease, other pulmonary diseases with mixed restrictive and obstructive pattern, sleep-disordered breathing, alveolar hypoventilation disorders, chronic exposure to high altitude, and developmental abnormalities;

WHO Group IV—chronic thromboembolic pulmonary hypertension (CTEPH); and WHO Group V—pulmonary hypertension with unclear multifactorial mechanisms, including hematologic diseases (e.g., myeloproliferative disease and splenectomy), systemic diseases (e.g., sarcoidosis, pulmonary Langerhans cell histiocytosis, lymphangioleiomyomatosis, neurofibromatosis and vasculitis), metabolic disorders (e.g., glycogen storage disease, Gaucher disease and thyroid diseases), and other causes (e.g., tumoral obstruction, fibrosing mediastinitis and chronic renal failure on dialysis).

The therapeutically effective amount and the frequency of administration of, and the length of treatment with, treprostinil or a salt thereof to treat, e.g., pulmonary hypertension may depend on various factors, including the type of pulmonary hypertension, the severity of the condition, the age, body weight, general health, gender and diet of the subject, and the response of the subject to the treatment, and can be determined by the treating physician. In some embodiments, the effective dose of treprostinil or a salt thereof per day is about 0.1-100 mg, 0.1-50 mg, 0.5-50 mg, 0.5-25 mg, 0.5-10 mg, 1-10 mg, 1-5 mg or 5-10 mg, or as deemed appropriate by the treating physician, which can be administered in a single dose or in divided doses. In certain embodiments, the effective dose of treprostinil or a salt thereof per day is about 0.1-10 mg, 0.1-5 mg, 0.5-5 mg or 1-5 mg. In further embodiments, the effective dose of treprostinil or a salt thereof per day is about 0.05 or 0.1 mg to 0.5 mg, 0.5-1 mg, 1-3 mg or 3-6 mg. In additional embodiments, the effective dose of treprostinil or a salt thereof per day is about 0.001-2 mg/kg, 0.005-1 mg/kg, 0.01-0.5 mg/kg or 0.01-0.1 mg/kg body weight, or as deemed appropriate by the treating physician.

In some embodiments, treprostinil or a salt thereof is administered, in a single dose or in multiple doses, daily (including one, two, three, four or more times daily), every two days, every three days, weekly, every 2 weeks, every 3 weeks, monthly, every 6 weeks, every 2 months or every 3 months, or as deemed appropriate by the treating physician. In certain embodiments, treprostinil or a salt thereof is administered daily. In further embodiments, treprostinil or a salt thereof is administered over a period of at least about 1 week, 2 weeks or 3 weeks. In other embodiments, treprostinil or a salt thereof is administered under a chronic dosing regimen. In certain embodiments, a therapeutically effective amount of treprostinil or a salt thereof is administered over a period of at least about 1 month (4 weeks), 6 weeks, 2 months, 3 months, 4 months, 5 months, 6 months, 1 year, 1.5 years, 2 years, 3 years or longer.

In some embodiments, treprostinil or a salt thereof is dermally or transdermally administered to treat PAH. In certain embodiments, an additional therapeutic agent is administered in combination with treprostinil or a salt thereof to treat PAH. The additional therapeutic agent can be administered concurrently with or sequentially to (before or after) administration of treprostinil or a salt thereof. If administered concurrently with treprostinil or a salt thereof, the additional therapeutic agent can be contained in the same composition as treprostinil or a salt thereof or in separate compositions.

In certain embodiments, the additional therapeutic agent for the treatment of pulmonary hypertension (e.g., PAH) is selected from:

vasoactive agents (including vasodilating agents), including without limitation prostaglandins and prostanoids (e.g., prostacyclin [prostaglandin I₂] and analogs thereof, such as beraprost, cicaprost and iloprost), other prostacyclin receptor agonists (e.g., selexipag and ACT-333679 [MRE-269]), calcium channel blockers (CCBs) (e.g., dihydropyridine-type CCBs [e.g., amlodipine and nifedipine] and non-dihydropyridine CCBs [e.g., diltiazem]), endothelin receptor (e.g., ETA or/and ETB) antagonists (e.g., ambrisentan, bosentan, sitaxentan and Actelion-1), phosphodiesterase type 5 (PDE5) inhibitors (e.g., avanafil, benzamidenafil, dynafil, lodenafil, mirodenafil, sildenafil, tadalafil, udenafil, vardenafil, dipyridamole, icariin, papaverine, propentofylline, zaprinast and T-1032), activators of soluble guanylate cyclase (e.g., cinaciguat and riociguat), and analogs, derivatives and salts thereof;

diuretics, including without limitation thiazide diuretics (e.g., bendroflumethiazide, chlorothiazide, epitizide and hydrochlorothiazide), thiazide-like diuretics (e.g., chlorthalidone, indapamide and metolazone), and analogs, derivatives and salts thereof;

anticoagulants, including without limitation vitamin K antagonists (e.g., acenocoumarol, atromentin, coumarin, phenindione, phenprocoumon and warfarin), direct thrombin inhibitors (e.g., argatroban, dabigatran, hirudin, lepirudin and bivalirudin), direct factor Xa inhibitors (e.g., apixaban, betrixaban, darexaban, edoxaban, eribaxaban, letaxaban and rivaroxaban), heparin and derivatives thereof (e.g., unfractionated heparin, low molecular weight heparin, fondaparinux and idraparinux), others (e.g., antithrombin, batroxobin and hementin), and analogs, derivatives, fragments and salts thereof; and other kinds of therapeutic agents, including without limitation cardiac glycosides (e.g., digoxin, acetyldigoxin, digoxigenin and digitoxin) and oxygen therapy.

### IX. REPRESENTATIVE EMBODIMENTS

The following embodiments of the disclosure are provided by way of example only:

1. A transdermal drug-delivery system (TDS) comprising treprostinil or a pharmaceutically acceptable salt, solvate, hydrate, clathrate or polymorph thereof, and one or more pharmaceutically acceptable excipients or carriers, wherein the TDS is formulated or configured to transdermally deliver treprostinil or a salt thereof to a subject.
2. The TDS of embodiment 1, which is formulated or configured for application to the surface of the skin.
3. The TDS of embodiment 1 or 2, which is formulated or configured to deliver treprostinil or a salt thereof into the blood for systemic distribution.
4. The TDS of any one of embodiments 1 to 3, which is a topical composition (e.g., an oil, a spray, a gel, a jelly, a liniment, a lotion, a cream, a foam, an ointment, a paste or a dressing) formulated for application to the skin.
5. The TDS of embodiment 4, wherein the topical composition further comprises one or more chemical permeation enhancers (e.g., a surfactant [e.g., sodium laureth sulfate] or/and an aromatic compound [e.g., 1-phenylpiperazine], or a fatty acid ester [e.g., isopropyl myristate] or/and an alcohol [e.g., ethanol]).
6. The TDS of any one of embodiments 1 to 3, which is a transdermal patch.
7. The TDS of embodiment 6, wherein the transdermal patch is a reservoir-type transdermal patch (RTP) comprising a liquid- or gel-based drug reservoir and optionally a semi-permeable membrane.
8. The TDS of embodiment 6, wherein the transdermal patch is a matrix-type transdermal patch (MTP) comprising a drug/polymer matrix.
9. The TDS of embodiment 8, wherein the drug/polymer matrix is not in an adhesive layer or is separate from an adhesive layer (e.g., a skin-contacting adhesive layer).
10. The TDS of embodiment 9, wherein the drug/polymer matrix comprises a plurality of microscopic drug reservoirs (drug microreservoirs).
11. The TDS of embodiment 9, wherein the drug/polymer matrix comprises a plurality of polymer-coated drug microparticles.
12. The TDS of any one of embodiments 9 to 11, wherein the transdermal patch further comprises a semi-permeable membrane.
13. The TDS of embodiment 8, wherein the drug/polymer matrix is part of an adhesive layer (e.g., part of a skin-contacting adhesive layer in a single-layer drug-in-adhesive [DIA] patch, part of a drug-loaded adhesive layer in a patch with a non-drug-loaded, skin-contacting, rate-controlling adhesive layer, part of each drug-loaded adhesive layer in a multi-layer, drug-gradient DIA patch, or part of a skin-contacting adhesive layer and part of a second adhesive layer with or without a semi-permeable membrane or other release-limiting layer between the two drug-loaded adhesive layers in a multi-layer DIA patch).
14. The TDS of embodiment 6, wherein the transdermal patch comprises solid microneedles or hollow microneedles.
15. The TDS of embodiment 14, wherein the solid microneedles are coated with treprostinil or a salt thereof, or the solid microneedles are composed of a bioabsorbable polymeric material containing treprostinil or a salt thereof.
16. The TDS of embodiment 14, wherein the transdermal patch comprising hollow microneedles further comprises a liquid-based drug reservoir.
17. The TDS of any one of embodiments 6 to 16, wherein the transdermal patch further comprises one or more chemical permeation enhancers (e.g., a fatty acid ester [e.g., isopropyl myristate] or/and an alcohol [e.g., ethanol], or a surfactant [e.g., sodium laureth sulfate] or/and an aromatic compound [e.g., 1-phenylpiperazine]).
18. The TDS of any one of embodiments 6 to 17, wherein the transdermal patch delivers a therapeutically effective amount of treprostinil or a salt thereof for up to about 72 hours (3 days) or 1 week (7 days).
19. The TDS of any one of embodiments 6 to 18, wherein the transdermal patch delivers a therapeutically effective amount of treprostinil or a salt thereof of from about 0.05 or 0.1 mg to about 0.5 mg, about 0.5-1 mg or about 1-5 mg per day.
20. The TDS of any one of embodiments 6 to 19, wherein the transdermal patch is a single transdermal patch, or is a patch unit among a plurality of patch units of a parent/mother patch that is divisible into a plurality of separate patch units (e.g., along lines of separation).
21. The TDS of any one of the preceding embodiments, which is integrated with a chemical or physical enhancement technique that provides an added driving force for drug transport into or/and through the skin (e.g., iontophoresis).
22. The TDS of any one of the preceding embodiments, which is integrated with a chemical or physical enhancement technique that increases skin permeability (e.g., chemical permeation enhancer, non-cavitational or cavitational ultrasound, electroporation, thermal ablation, radio frequency, microdermabrasion, microneedles or high pressure).
23. The TDS of any one of the preceding embodiments, wherein the treprostinil is the carboxylic acid form of treprostinil.
24. The TDS of any one of embodiments 1 to 22, wherein the treprostinil is a salt form of treprostinil, such as an alkali metal salt (e.g., treprostinil sodium) or an amine salt (e.g., treprostinil diethanolamine, treprostinil triethanolamine, treprostinil 2-amino-2-methyl-1,3-propanediol or treprostinil tris(hydroxymethyl)aminomethane).
25. The TDS of any one of the preceding embodiments, further comprising an additional therapeutic agent.
26. The TDS of embodiment 25, wherein the additional therapeutic agent is selected from local anesthetics, analgesics, anti-inflammatory agents, local vasoconstrictors, and combinations thereof.
27. A method of transdermally delivering treprostinil or a salt thereof to a subject, comprising applying the transdermal drug-delivery system of any one of the preceding embodiments to the skin of the subject.
28. A method of treating a medical condition responsive to treatment with treprostinil, comprising transdermally administering a therapeutically effective amount of treprostinil or a pharmaceutically acceptable salt, solvate, hydrate, clathrate or polymorph thereof to a subject in need of treatment.
29. The method of embodiment 28, wherein treprostinil or a salt thereof is delivered into the blood for systemic distribution.
30. The method of embodiment 28 or 29, wherein the transdermally administering treprostinil or a salt thereof to the subject comprises applying the transdermal drug-delivery system (TDS) of any one of embodiments 1 to 26 to the skin of the subject.
31. The method of any one of embodiments 28 to 30, wherein treprostinil or a salt thereof is administered via a transdermal patch.
32. The method of embodiment 30 or 31, further comprising administering an additional therapeutic agent locally at or near the site of application of the TDS or the transdermal patch.
33. The method of embodiment 32, wherein the additional therapeutic agent is selected from local anesthetics, analgesics, anti-inflammatory agents, local vasoconstrictors, and combinations thereof.
34. The method of any one of embodiments 28 to 33, wherein the medical condition is selected from pulmonary hypertension, pulmonary fibrosis, interstitial lung disease, asthma, congestive heart failure, peripheral vascular disease, severe intermittent claudication, atherogenesis, ischemic lesions, critical limb ischemia, ischemic ulcers, skin ulcers, neuropathic foot ulcers, kidney malfunction and failure, immunosuppression, proliferative disorders, and pain associated with each of the preceding conditions.
35. The method of embodiment 34, wherein the medical condition is pulmonary hypertension, such as pulmonary arterial hypertension.
36. The method of any one of embodiments 28 to 35, further comprising administering an additional therapeutic agent.
37. The method of embodiment 36, wherein the additional therapeutic agent is selected from vasoactive agents, diuretics, anticoagulants, cardiac glycosides, and combinations thereof.
38. The method of any one of embodiments 28 to 37, wherein the therapeutically effective amount of treprostinil or a salt thereof is from about 0.05 or 0.1 mg to about 0.5 mg, about 0.5-1 mg or about 1-5 mg per day.
39. Treprostinil or a pharmaceutically acceptable salt, solvate, hydrate, clathrate or polymorph thereof for use in the treatment of a medical condition responsive to treatment with treprostinil, wherein treprostinil or a salt thereof is transdermally administered.
40. A composition comprising treprostinil or a pharmaceutically acceptable salt, solvate, hydrate, clathrate or polymorph thereof for use in the treatment of a medical condition responsive to treatment with treprostinil, wherein treprostinil or a salt thereof is transdermally administered.
41. Use of treprostinil or a pharmaceutically acceptable salt, solvate, hydrate, clathrate or polymorph thereof in the preparation of a medicament for the treatment of a medical condition responsive to treatment with treprostinil, wherein treprostinil or a salt thereof is transdermally administered.
42. The compound, composition or use of embodiment 39, 40 or 41, respectively, wherein treprostinil or a salt thereof is transdermally administered via the transdermal drug-delivery system of any one of embodiments 1 to 26.
43. The compound, composition or use of any one of embodiments 39 to 42, wherein treprostinil or a salt thereof is administered via a transdermal patch.
44. The compound, composition or use of any one of embodiments 39 to 43, wherein the medical condition is pulmonary hypertension, such as pulmonary arterial hypertension.
45. A kit comprising the transdermal drug-delivery system (TDS) of any one of embodiments 1 to 26, and instructions for using the TDS to treat a medical condition responsive to treatment with treprostinil.
46. The kit of embodiment 45, wherein the TDS is a transdermal patch.
47. The kit of embodiment 45 or 46, wherein the medical condition is pulmonary hypertension, such as pulmonary arterial hypertension.

### X. EXAMPLES

The following examples are intended only to illustrate the disclosure. Other assays, studies, protocols, procedures, methodologies, reagents and conditions may alternatively be used as appropriate.

### Example 1. In Vitro Skin Permeability Assay of Treprostinil

A skin permeability assay was performed using a vertical Franz diffusion cell having a diffusion area of 0.64 cm² and a volume of 7.5 mL. The assay was conducted at 32° C. with continuous stirring. Heat-separated human cadaver epidermis was used in the assay, the epidermis being stored at −20° C. after the heat-stripping procedure. The human epidermis was thawed prior to being mounted on the diffusion cell. Treprostinil was applied on the skin, and the diffusion cell was closed by screw-cap. At various time intervals, whole medium or receptor medium was replaced by fresh medium. Part of the collected medium was used to calculate the cumulative skin permeability of treprostinil at 72 hours. The skin permeability of treprostinil was evaluated using human epidermis from different donors. N=4 replicates were performed for treprostinil tested on human epidermis from a particular donor. Treprostinil exhibited high cumulative skin permeability at 72 hours when tested on human epidermis from different donors.

### Example 2. Skin Irritation Study of Treprostinil in Minipigs

The pig is frequently used in studies involving dermal or transdermal administration of a drug because the skin of the pig is very similar to human skin.

Skin irritation following transdermal administration of treprostinil from a reservoir-type transdermal patch was assessed in Göttingen Minipigs® (Marshall BioResources, North Rose, N.Y.). The treprostinil-containing reservoir transdermal patch was a 32 cm² circular patch with a 6 cm² gel-sealed area and a 26 cm² peripheral skin-adhesive area. The placebo reservoir transdermal patch was a 39 cm² oval patch with a 12 cm² gel-sealed area and a 27 cm² peripheral skin-adhesive area. The reservoir of the patches comprised a gel composed of hydroxypropyl cellulose and containing ethanol, isopropyl myristate and 10 mg treprostinil or no active agent for the placebo patch. The reservoir was packaged by heat seal between a backing layer composed of metalized polyester/ethylene-vinyl acetate and a semi-permeable microporous membrane composed of polyethylene. A transparent release liner that protected the gel reservoir area and the peripheral adhesive area was peeled off immediately prior to patch application. The patches had the structure of the patch depicted in FIG. 1.

To facilitate patch application (dosing) and to ensure animal safety during the dosing procedure, the minipigs were sedated with the anesthetic Telazol, if necessary, via intramuscular injection at an initial dose of 3-6 mg/kg. Any use of Telazol is indicated in the study data.

Both the treprostinil-containing and placebo transdermal patches were applied to the dorsal surface of each of three female Göttingen Minipigs® weighing about 15-25 kg and were left in place for 24 hr. The dosing sites (the entire flank on both sides of the dorsal midline, caudal to the scapula) were carefully clipped with an electric clipper on the day prior to application of the patches and then were carefully washed with room temperature (RT) saline to remove dirt and debris. The prepared area was large enough to accommodate several patches. The dosing sites then were carefully shaved smooth with a razor while not causing any nick or cut. RT saline, but no shaving cream, could be used to assist shaving. The dosing sites were then wrapped with bandaging that was secured with a non-irritating, semi-occlusive tape (e.g., Elastikon tape). The minipigs were then fitted with a jacket. On the day of application of the patches, the jacket and wrapping were removed from the minipigs. The dosing sites were visually inspected to confirm the absence of any abrasion or obvious dermal pathology, and were gently cleaned with an isopropanol wipe and were then allowed to air-dry for at least 5 min. The patches were applied to areas of the skin without any abrasion or obvious dermal pathology by applying firm finger pressure to the peripheral adhesive area of the patches to ensure firm adherence of the peripheral area to the skin. To prevent detachment of the patches, Tegaderm™ dressing (or an equivalent transparent adhesive dressing, such as PatchProtect™ dressing) was applied over the patches, extending at least 2 inches beyond the edges of the patches and adhering to the peripheral area of the patches and to the skin surrounding the patches. The sites of patch application were wrapped with gauze or Vetrap bandaging (or equivalent) that was secured with a non-irritating, semi-occlusive tape (e.g., Elastikon tape). The minipigs were then fitted with a jacket to prevent patch disturbance. The patches were kept in place at the dosing sites for 24 hr.

The sites of transdermal administration were observed for gross signs of irritation (erythema, edema and lesion) and any other signs of local or systemic effect, prior to patch application and at 2 (±15 min), 24 (±1 hr), 48, 72, 96, 120, 144 and 168 hours after patch removal. Photographs of the sites of patch application were taken just prior to patch removal, following patch removal, and at 2 (±15 min), 24 (±1 hr), 48, 72, 96, 120, 144 and 168 hours after patch removal.

The following scores are based on the Draize scale for scoring skin irritation [J. Draize et al., J. Pharmacol. Exp. Ther., 82:377-390 (1944)].

Erythema and Eschar Formation

| Score | Classification |
|---|---|
| 0 | No erythema |
| 1 | Very slight erythema (barely perceptible) |
| 2 | Well-defined erythema |
| 3 | Moderate to severe erythema |
| 4 | Severe erythema (beet redness) to slight |
| | eschar formation (injuries in depth) |

Edema Formation

| Score | Classification |
|---|---|
| 0 | No edema |
| 1 | Very slight edema (barely perceptible) |
| 2 | Slight edema (edges of area well defined by definite raising) |
| 3 | Moderate edema (raised about 1 mm) |
| 4 | Severe edema (raised >1 mm and extending beyond area of |
| | exposure) |

Table 1 shows the erythema and edema scores for the treprostinil-containing and placebo transdermal patches at various time points. The three Göttingen Minipigs® used in the study are numbered 601, 602 and 603. As can be seen from Table 1, delivery of treprostinil as well as the chemical permeation enhancers isopropyl myristate and ethanol from the transdermal patch did not cause significant skin irritation, causing only slight or mild erythema in one (numbered 603) of the three minipigs.

TABLE 1

| | | Erythema | | | Edema | | |
|---|---|---|---|---|---|---|---|
| Patch | Time Point | 601 | 602 | 603 | 601 | 602 | 603 |
| Treprostinil | Before patch | 0 | 0 | 0 | 0 | 0 | 0 |
| | application | | | | | | |
| | 2 hr post | 1 | 1 | 2 | 0 | 0 | 0 |
| | patch removal | | | | | | |
| | 24 hr post | 1 | 1 | 2 | 0 | 0 | 0 |
| | patch removal | | | | | | |
| | 48 hr post | 1 | 0 | 0 | 0 | 0 | 0 |
| | patch removal | | | | | | |
| | 72 hr post | 0 | 0 | 0 | 0 | 0 | 0 |
| | patch removal | | | | | | |
| | 96 hr post | 0 | 0 | 0 | 0 | 0 | 0 |
| | patch removal | | | | | | |
| | 120 hr post | 0 | 0 | 0 | 0 | 0 | 0 |
| | patch removal | | | | | | |
| Placebo | Before patch | 0 | 0 | 0 | 0 | 0 | 0 |
| | application | | | | | | |
| | 2 hr post patch | 1 | 0 | 1 | 0 | 0 | 0 |
| | removal | | | | | | |
| | 24 hr post | 1 | 0 | 1 | 0 | 0 | 0 |
| | patch removal | | | | | | |
| | 48 hr post | 1 | 0 | 1 | 0 | 0 | 0 |
| | patch removal | | | | | | |
| | 72 hr post | 0 | 0 | 0 | 0 | 0 | 0 |
| | patch removal | | | | | | |
| | 96 hr post | 1 | 0 | 0 | 0 | 0 | 0 |
| | patch removal | | | | | | |
| | 120 hr post | 1 | 1 | 0 | 0 | 0 | 0 |
| | patch removal | | | | | | |

### Example 3. Pharmacokinetics of Transdermally Delivered Treprostinil in Minipigs

Pharmacokinetics (PK) following transdermal administration of treprostinil from a reservoir-type transdermal patch was studied in Göttingen Minipigs® (Marshall BioResources). The treprostinil-containing reservoir transdermal patch was a 39 cm² oval patch with a 12 cm² gel-sealed area and a 27 cm² peripheral skin-adhesive area. The patch had a similar composition and structure as the treprostinil-containing patch described in Example 2, except that the reservoir of the patch used in the PK study contained 40 mg treprostinil.

Naïve male Göttingen Minipigs® about 6 months of age at receipt and weighing about 23 kg at patch application were used in the PK study. To facilitate application of the transdermal patch, the minipigs were sedated with Telazol at 3 mg/kg via intramuscular injection. Prior to patch application the dosing sites on the minipigs were prepared similarly as described in Example 2, and patch application was performed similarly as described in Example 2 except that bandaging was reapplied to the sites of patch application once a day. Four transdermal patches containing a total of 160 mg treprostinil were applied at the same time to the dorsal surface of each of two minipigs and were left in place for 72 hr. Blood samples were collected within 1 hr prior to application of the patches; at 3, 6, 12, 24, 48 and 72 hours after application of the patches; and at 0.167, 0.5, 1, 2, 4 and 6 hours after removal of the patches.

For the two minipigs, delivery of treprostinil from the transdermal patches resulted in a Cₘₐₓ of 20.8-27.6 ng/mL, a C₇₂ₕᵣ of 2.4-4.9 ng/mL, a Tₘₐₓ of 6 hr, an AUC₀₋₇₂ of 429-946 ng·hr/mL, and a half-life (T_{1/2}) during treatment of 28-35 hr.

It is understood that, while particular embodiments have been illustrated and described, various modifications may be made thereto and are contemplated herein. It is also understood that the disclosure is not limited by the specific examples provided herein. The description and illustration of embodiments and examples of the disclosure herein are not intended to be construed in a limiting sense. It is further understood that all aspects of the disclosure are not limited to the specific depictions, configurations or relative proportions set forth herein, which may depend upon a variety of conditions and variables. Various modifications and variations in form and detail of the embodiments and examples of the disclosure will be apparent to a person skilled in the art. It is therefore contemplated that the disclosure also covers any and all such modifications, variations and equivalents.

What is claimed is:

1. A transdermal drug-delivery system (TDS) comprising treprostinil or a pharmaceutically acceptable salt, solvate, hydrate, clathrate or polymorph thereof, and one or more pharmaceutically acceptable excipients or carriers, wherein the TDS is formulated or configured to transdermally deliver treprostinil or a salt thereof to a subject.

2. The TDS of claim 1, which is formulated or configured for application to the surface of the skin.

3. The TDS of claim 1 or 2, which is formulated or configured to deliver treprostinil or a salt thereof into the blood for systemic distribution.

4. The TDS of any one of claims 1 to 3, which is a topical composition (e.g., an oil, a spray, a gel, a jelly, a liniment, a lotion, a cream, a foam, an ointment, a paste or a dressing) formulated for application to the skin.

5. The TDS of claim 4, wherein the topical composition further comprises one or more chemical permeation enhancers (e.g., a surfactant [e.g., sodium laureth sulfate] or/and an aromatic compound [e.g., 1-phenylpiperazine], or a fatty acid ester [e.g., isopropyl myristate] or/and an alcohol [e.g., ethanol]).

6. The TDS of any one of claims 1 to 3, which is a transdermal patch.

7. The TDS of claim 6, wherein the transdermal patch is a reservoir-type transdermal patch (RTP) comprising a liquid- or gel-based drug reservoir and optionally a semi-permeable membrane.

8. The TDS of claim 6, wherein the transdermal patch is a matrix-type transdermal patch (MTP) comprising a drug/polymer matrix.

9. The TDS of claim 8, wherein the drug/polymer matrix is not in an adhesive layer or is separate from an adhesive layer (e.g., a skin-contacting adhesive layer).

10. The TDS of claim 9, wherein the drug/polymer matrix comprises a plurality of microscopic drug reservoirs (drug microreservoirs).

11. The TDS of claim 9, wherein the drug/polymer matrix comprises a plurality of polymer-coated drug microparticles.

12. The TDS of any one of claims 9 to 11, wherein the transdermal patch further comprises a semi-permeable membrane.

13. The TDS of claim 8, wherein the drug/polymer matrix is part of an adhesive layer (e.g., part of a skin-contacting adhesive layer in a single-layer drug-in-adhesive [DIA] patch, part of a drug-loaded adhesive layer in a patch with a non-drug-loaded, skin-contacting, rate-controlling adhesive layer, part of each drug-loaded adhesive layer in a multi-layer, drug-gradient DIA patch, or part of a skin-contacting adhesive layer and part of a second adhesive layer with or without a semi-permeable membrane or other release-limiting layer between the two drug-loaded adhesive layers in a multi-layer DIA patch).

14. The TDS of claim 6, wherein the transdermal patch comprises solid microneedles or hollow microneedles.

15. The TDS of claim 14, wherein the solid microneedles are coated with treprostinil or a salt thereof, or the solid microneedles are composed of a bioabsorbable polymeric material containing treprostinil or a salt thereof.

16. The TDS of claim 14, wherein the transdermal patch comprising hollow microneedles further comprises a liquid-based drug reservoir.

17. The TDS of any one of claims 6 to 16, wherein the transdermal patch further comprises one or more chemical permeation enhancers (e.g., a fatty acid ester [e.g., isopropyl myristate] or/and an alcohol [e.g., ethanol], or a surfactant [e.g., sodium laureth sulfate] or/and an aromatic compound [e.g., 1-phenylpiperazine]).

18. The TDS of any one of claims 6 to 17, wherein the transdermal patch delivers a therapeutically effective amount of treprostinil or a salt thereof for up to about 72 hours (3 days) or 1 week (7 days).

19. The TDS of any one of claims 6 to 18, wherein the transdermal patch delivers a therapeutically effective amount of treprostinil or a salt thereof of from about 0.05 or 0.1 mg to about 0.5 mg, about 0.5-1 mg or about 1-5 mg per day.

20. The TDS of any one of claims 6 to 19, wherein the transdermal patch is a single transdermal patch, or is a patch unit among a plurality of patch units of a parent/mother patch that is divisible into a plurality of separate patch units (e.g., along lines of separation).

21. The TDS of any one of the preceding claims, which is integrated with a chemical or physical enhancement technique that provides an added driving force for drug transport into or/and through the skin (e.g., iontophoresis).

22. The TDS of any one of the preceding claims, which is integrated with a chemical or physical enhancement technique that increases skin permeability (e.g., chemical permeation enhancer, non-cavitational or cavitational ultrasound, electroporation, thermal ablation, radio frequency, microdermabrasion, microneedles or high pressure).

23. The TDS of any one of the preceding claims, wherein the treprostinil is the carboxylic acid form of treprostinil.

24. The TDS of any one of claims 1 to 22, wherein the treprostinil is a salt form of treprostinil, such as an alkali metal salt (e.g., treprostinil sodium) or an amine salt (e.g., treprostinil diethanolamine, treprostinil triethanolamine, treprostinil 2-amino-2-methyl-1,3-propanediol or treprostinil tris(hydroxymethyl)aminomethane).

25. The TDS of any one of the preceding claims, further comprising an additional therapeutic agent.

26. The TDS of claim 25, wherein the additional therapeutic agent is selected from local anesthetics, analgesics, anti-inflammatory agents, local vasoconstrictors, and combinations thereof.

27. A method of transdermally delivering treprostinil or a salt thereof to a subject, comprising applying the transdermal drug-delivery system of any one of the preceding claims to the skin of the subject.

28. A method of treating a medical condition responsive to treatment with treprostinil, comprising transdermally administering a therapeutically effective amount of treprostinil or a pharmaceutically acceptable salt, solvate, hydrate, clathrate or polymorph thereof to a subject in need of treatment.

29. The method of claim 28, wherein treprostinil or a salt thereof is delivered into the blood for systemic distribution.

30. The method of claim 28 or 29, wherein the transdermally administering treprostinil or a salt thereof to the subject comprises applying the transdermal drug-delivery system (TDS) of any one of claims 1 to 26 to the skin of the subject.

31. The method of any one of claims 28 to 30, wherein treprostinil or a salt thereof is administered via a transdermal patch.

32. The method of claim 30 or 31, further comprising administering an additional therapeutic agent locally at or near the site of application of the TDS or the transdermal patch.

33. The method of claim 32, wherein the additional therapeutic agent is selected from local anesthetics, analgesics, anti-inflammatory agents, local vasoconstrictors, and combinations thereof.

34. The method of any one of claims 28 to 33, wherein the medical condition is selected from pulmonary hypertension, pulmonary fibrosis, interstitial lung disease, asthma, congestive heart failure, peripheral vascular disease, severe intermittent claudication, atherogenesis, ischemic lesions, critical limb ischemia, ischemic ulcers, skin ulcers, neuropathic foot ulcers, kidney malfunction and failure, immunosuppression, proliferative disorders, and pain associated with each of the preceding conditions.

35. The method of claim 34, wherein the medical condition is pulmonary hypertension, such as pulmonary arterial hypertension.

36. The method of any one of claims 28 to 35, further comprising administering an additional therapeutic agent.

37. The method of claim 36, wherein the additional therapeutic agent is selected from vasoactive agents, diuretics, anticoagulants, cardiac glycosides, and combinations thereof.

38. The method of any one of claims 28 to 37, wherein the therapeutically effective amount of treprostinil or a salt thereof is from about 0.05 or 0.1 mg to about 0.5 mg, about 0.5-1 mg or about 1-5 mg per day.

39. Treprostinil or a pharmaceutically acceptable salt, solvate, hydrate, clathrate or polymorph thereof for use in the treatment of a medical condition responsive to treatment with treprostinil, wherein treprostinil or a salt thereof is transdermally administered.

40. A composition comprising treprostinil or a pharmaceutically acceptable salt, solvate, hydrate, clathrate or polymorph thereof for use in the treatment of a medical condition responsive to treatment with treprostinil, wherein treprostinil or a salt thereof is transdermally administered.

41. Use of treprostinil or a pharmaceutically acceptable salt, solvate, hydrate, clathrate or polymorph thereof in the preparation of a medicament for the treatment of a medical condition responsive to treatment with treprostinil, wherein treprostinil or a salt thereof is transdermally administered.

42. The compound, composition or use of claim 39, 40 or 41, respectively, wherein treprostinil or a salt thereof is transdermally administered via the transdermal drug-delivery system of any one of claims 1 to 26.

43. The compound, composition or use of any one of claims 39 to 42, wherein treprostinil or a salt thereof is administered via a transdermal patch.

44. The compound, composition or use of any one of claims 39 to 43, wherein the medical condition is pulmonary hypertension, such as pulmonary arterial hypertension.

45. A kit comprising the transdermal drug-delivery system (TDS) of any one of claims 1 to 26, and instructions for using the TDS to treat a medical condition responsive to treatment with treprostinil.

46. The kit of claim 45, wherein the TDS is a transdermal patch.

47. The kit of claim 45 or 46, wherein the medical condition is pulmonary hypertension, such as pulmonary arterial hypertension.
